SEQUENCE LISTING (6 sequences)

SEQ ID NO: 1 - DNA, 30 bp, Artificial Sequence
  Toxin A forward primer
tgatgctaataatgaatctaaaatggtaac

SEQ ID NO: 2 - DNA, 19 bp, Artificial Sequence
  Toxin A reverse primer
accaccagctgcagccata

SEQ ID NO: 3 - DNA, 28 bp, Artificial Sequence
  Toxin B forward primer
gtgtagcaatgaaagtccaagtttacgc

SEQ ID NO: 4 - DNA, 26 bp, Artificial Sequence
  Toxin B reverse primer
cacttagctctttgattgctgcacct

SEQ ID NO: 5 - DNA, 20 bp, Artificial Sequence
  16S rRNA forward primer
acacggtccaaactcctacg

SEQ ID NO: 6 - DNA, 20 bp, Artificial Sequence
  16S rRNA reverse primer
aggcgagtttcagcctacaa

(19) United States
(12) Patent Application Publication
(10) Pub. No.: US 2015/0315627 A1
(43) Pub. Date: Nov. 5, 2015

(54) METHODS AND COMPOSITIONS FOR THE DETECTION OF FUNCTIONAL CLOSTRIDIUM DIFFICILE TOXINS

(71) Applicant: BOARD OF REGENTS OF THE UNIVERSITY OF TEXAS SYSTEM, Austin, TX (US)

(72) Inventors: Charles DARKOH, Houston, TX (US); Herbert L. DUPONT, Houston, TX (US); Heidi B. KAPLAN, Houston, TX (US)

(21) Appl. No.: 14/123,197

(22) PCT Filed: May 31, 2012
(86) PCT No.: PCT/US2012/040089

§ 371 Date: Nov. 12, 2014

Related U.S. Application Data
(60) Provisional application No. 61/491,726, filed on May 31, 2011.

(51) Int. Cl. C12Q 1/48

(52) U.S. Cl. CPC C12Q 1/48; G01N 2500/04; G01N 2333/91097

(57) ABSTRACT

Methods and compositions for the identification of functional C. difficile toxin, as among other things identifying individuals infected with toxigenic C. difficile and therefore in need of therapy. In specific embodiments, the methods and compositions provide colorimetric assays for cleavage activity of C. difficile toxin.

### CROSS-REFERENCE TO RELATED APPLICATIONS

The present application claims priority to provisional patent application No. 61/491,726 filed May 31, 2011, entitled “Methods and Compositions for the Detection of Functional Clostridium Difficile Toxins.”

### STATEMENT REGARDING FEDERALLY SPONSORED RESEARCH OR DEVELOPMENT

This invention was made with U.S. Government support under Grant No. A1055449 awarded by the National Institutes of Health. The government has certain rights in the invention.

### TECHNICAL FIELD

This disclosure relates to the detection of functional C. difficile toxins by identifying the presence of toxigenic strains of C. difficile, and to identification of active C. difficile toxins to aid in, among other things, diagnosis and indications for therapy.

### BACKGROUND

Clostridium difficile is the leading identifiable cause of nosocomial diarrhea worldwide due to its virulence, multi-drug resistance, spore-forming ability, and environmental persistence (Bartlett, J. G. 1992. Antibiotic-associated diarrhea. Clin Infect Dis 15:573-581. McDonald, L. et al., 2005. An epidemic, toxin gene-variant strain of Clostridium difficile. N Engl J Med 353:2433-2441; Poutanen, S. M. et al., 2004. Clostridium difficile-associated diarrhea in adults. CMAJ 171:51-58; Warny, M., J. et al., 2005. Toxin production by an emerging strain of Clostridium difficile associated with outbreaks of severe disease in North America and Europe. Lancet 366:1079-1084). This bacterium has been implicated as the causative organism for 10-25% of the reported cases of antibiotic-associated diarrhea, 50-75% of antibiotic-associated colitis, and 90-100% of pseudomembranous colitis (Bartlett, J. G. 2002. Clinical practice. Antibiotic-associated diarrhea. N Engl J Med 346:334-339; Warny, et al., 2005, ibid). The toxigenic strains of C. difficile possess a 19.6 kb pathogenicity locus that encodes two notable proteins: toxins A (308 kDa) and B (269 kDa). These toxins are important virulent factors in the pathogenesis of C. difficile (Geric, B. M. et al., 2004. Distribution of Clostridium difficile variant toxinotypes and strains with binary toxin genes among clinical isolates in an American hospital. J Med Microbiol 53:887-894; Kuehne, S. A., et al., 2010. The role of toxin A and toxin B in Clostridium difficile infection. Nature 7; 467 (7316):711-3; Lyerly, D. M. et al., 1985. Effects of Clostridium difficile toxins given intragastrically to animals. Infect Immun 47:349-352; Rupnik, M., et al., 2001. Comparison of toxinotyping and PCR ribotyping of Clostridium difficile strains and description of novel toxinotypes. Microbiology 147:439-447; Voth, D. E., and J. D. Ballard. 2005. Clostridium difficile toxins: mechanism of action and role in disease. Clin Microbiol Rev 18:247-263). Both toxins have the same enzymatic cleavage activity (Dillon, S. T., et al., 1995. Involvement of Ras-related Rho proteins in the mechanisms of action of Clostridium difficile toxin A and toxin B. Infect Immun 63:1421-1426; Just, I., J. et al., 1995. Glucosylation of Rho proteins by Clostridium difficile toxin B. Nature 375:500-503; Just, I., M. Wilm, J. Selzer, G. Rex, C. von Eichel-Streiber, M. Mann, and K. Aktories. 1995. The enterotoxin from Clostridium difficile (ToxA) monoglucosylates the Rho proteins. J Biol Chem 270:13932-13936) and are cytotoxic to cultured cells, however, toxin B is 100 to 1,000-fold more potent than toxin A in most cell lines (Just, I., and R. Gerhard. 2004. Large clostridial cytotoxins. Rev Physiol Biochem Pharmacol 152:23-47; von Eichel-Streiber, C., P. et al., 1996. Large clostridial cytotoxins—a family of glycosyltransferases modifying small GTP-binding proteins. Trends Microbiol 4:375-382; Voth, et al., 2005, ibid).

The C-terminus of these toxins has a β-solenoid structure that is involved in receptor binding (Hofmann F, et al., 1997. Localization of the glucosyltransferase activity of Clostridium difficile toxin B to the N-terminal part of the holotoxin. J Biol Chem 272:11074-8; Sung J Y, et al., 1993. Antibacterial activity of bile salts against common biliary pathogens. Effects of hydrophobicity of the molecule and in the presence of phospholipids. Dig Dis Sci 38:2104-12). The central regions of the toxins possess a cysteine protease activity, which cleaves the N-terminal region in the presence of inositol hexakisphosphate, releasing the N-terminally located glucosyltransferase domain into the cytosol of the mammalian host (Egerer, M., T. et al., 2007. Auto-catalytic cleavage of Clostridium difficile toxins A and B depends on cysteine protease activity. J Biol Chem 282:25314-25321. Hofmann, F., C. et al., 1997. Localization of the glucosyltransferase activity of Clostridium difficile toxin B to the N-terminal part of the holotoxin. J Biol Chem 272:11074-11078; Pfeifer, G., J. et al., 2003. Cellular uptake of Clostridium difficile toxin B. Translocation of the N-terminal catalytic domain into the cytosol of eukaryotic cells. J Biol Chem 278:44535-44541. Rupnik, M., S. et al., 2005. Characterization of the cleavage site and function of resulting cleavage fragments after limited proteolysis of Clostridium difficile toxin B (TcdB) by host cells. Microbiology 151:199-208). The glucosyltransferase domain monoglucosylates low molecular weight GTPases of the Rho family (RhoA, B, C, Rac, and Cdc42) in the host cytosol using cellular uridine diphosphoglucose (UDP-glucose) as the glucose donor (Just, I., and R. Gerhard. 2004. Large clostridial cytotoxins. Rev Physiol Biochem Pharmacol 152:23-47; Just, I., et al., 1995. Glucosylation of Rho proteins by Clostridium difficile toxin B. Nature 375:500-503. This monoglucosylation interrupts the normal function of the Rho GTPases leading to a variety of effects on intoxicated cells such as apoptosis, cell rounding, actin cytoskeleton dysregulation, and altered cellular signaling (Genth, H., et al., 2008. Clostridium difficile toxins: more than mere inhibitors of Rho proteins. Int J Biochem Cell Biol 40:592-597; Hofmann, F et al., 1997, ibid; Just, I., and R. Gerhard. 2004, ibid; Just, I., et al., 1995, ibid).

Currently, only one non-radioactive assay (the tissue culture cytotoxicity assay) is available for the detection of the activities of the toxins. However, quantitative analysis of toxin activity using this method is tedious and requires the maintenance of a tissue-culture system, which makes it costly in terms of time and effort.

Current clinical identification of C. difficile in fecal samples relies on a combination of at least two techniques, which may include culture isolation, PCR detection of the toxin-encoding genes, the tissue culture cytotoxicity assay, and immunological detection of the toxins (ELISA). Culture isolation is normally performed on the commercially available media, cycloserine-cefoxitin fructose agar (CCFA), which is selective but does not differentiate the toxin-producing strains. As a result, a second method is required to determine if a strain is pathogenic. PCR assays are gaining popularity for the diagnosis of CDI because of their high sensitivity in detecting the toxin-encoding genes. The tissue culture cytotoxicity method is not as sensitive as culture isolation combined with toxin testing (Bartlett, J. G. 2002, ibid; Choy, F. Y., and R. G. Davidson. 1980. Gaucher's disease II. Studies on the kinetics of beta-glucosidase and the effects of sodium taurocholate in normal and Gaucher tissues. Pediatr Res 14:54-59; Dillon, S. T., et al., 1995), although it is considered by some laboratories as the gold standard. Other approaches include the glutamate dehydrogenase screening assay (McDonald, L. C., et al., 2005, ibid; Peters, S. P., et al., 1976. Differentiation of beta-glucocerebrosidase from beta-glucosidase in human tissues using sodium taurocholate. Arch Biochem Biophys 175:569-582), and automated PCR-based methods such as Cepheid Xpert Clostridium difficile Epi Assay (Bachmair, A., et al., 1986. In vivo half-life of a protein is a function of its amino-terminal residue. Science 234:179-186; Muto, C. A., et al., 2005. A large outbreak of Clostridium difficile-associated disease with an unexpected proportion of deaths and colectomies at a teaching hospital following increased fluoroquinolone use Infect Control Hosp Epidemiol 26:273-280), and the loop-mediated isothermal amplification test (Lanis, J. M., et al., 2010. Variations in TcdB activity and the hypervirulence of emerging strains of Clostridium difficile. PLoS Pathog 6). However, these methods do not isolate and differentiate toxigenic from non-toxigenic strains of C. difficile.

### SUMMARY

The presently disclosed compositions and methods are based, in part, on the discovery that the A and B toxins of C. difficile cleave indicator-linked substrates that have stereochemical characteristics similar to their natural substrate, UDP-glucose. In accordance with certain embodiments, methods and compositions for detection of functional C. difficile toxins are disclosed. In some embodiments, a quantitative assay (Cdifftox Activity assay) is provided that enables, in many cases, cost-efficient, sensitive, quantitative measurement of the cleavage activities of toxins A and B of C. difficile in both a culture supernatant and a selective and differential agar-based assay. The disclosed Cdifftox Plate assay (CDPA) enables identification of toxin-producing C. difficile without the need for additional toxin-confirmatory tests. These and other embodiments, features and advantages will be apparent in the drawings and in the following description.

### BRIEF DESCRIPTION OF THE DRAWINGS

FIG. 1 depicts the elution profile of the proteins in C. difficile culture supernatant separated by DEAE-Sepharose anion exchange chromatography. Fractions (10 ml) were examined using the Cdifftox Activity assay (A, top chart) and the antibody-based ELISA assay (B, lower chart). The Cdifftox Activity assay was performed by incubating 200 μl of each fraction in 50 mM Tris-HCl containing 50 mM NaCl (pH 7.4) with PNPG substrate reagent at 37° C. for 4 hrs. The assay was monitored by measuring absorbance at 410 nm. The protein concentration was determined using Bradford protein assay (BioRad). The ELISA assay was performed using the Wampole C. difficile TOX A/B II assay (TechLab, Blacksburg, Va.).

FIG. 2 depicts the elution profile of the pooled C. difficile toxin-positive fractions purified by Sephacryl S-300 gel filtration chromatography. Fractions (5 ml) were examined using the Cdifftox Activity assay (A, top chart) and the antibody-based ELISA assay (B, bottom chart). The Cdifftox Activity assay was performed by incubating 200 μl of each fraction in 50 mM Tris-HCl containing 50 mM NaCl (pH 7.4) with PNPG substrate reagent at 37° C. for 4 hrs. The assay was monitored by measuring absorbance at 410 nm. The protein concentration was determined using Bradford protein assay (BioRad). The ELISA assay was performed using the Wampole C. difficile TOX A/B II assay (TechLab, Blacksburg, Va.).

FIG. 3A depicts a polyacrylamide gel electrophoresis (PAGE) analysis of C. difficile toxins A and B purification by anion exchange and gel filtration chromatography. Proteins (50 μg each) were separated through a 5% PAGE gel. M=ProSieve QuadColor molecular weight marker (Lonza Rockland Inc., ME); 1=concentrated supernatant; 2=pooled and concentrated fractions from anion exchange; 3=pooled and concentrated fractions from gel filtration (toxin A); 4=pooled and concentrated fractions from gel filtration (toxin B). The arrow indicates the location of toxins in the gel.

FIG. 3B depicts a Western immunoblot analysis of C. difficile toxins A and B after gel filtration chromatography. Proteins (85 μg each) were separated by electrophoresis through a 5% PAGE gel and transferred onto PVDF membranes. Each membrane was probed using mouse monoclonal primary antibodies specific for toxins A or B. The WesternDot 625 Western Blot kit (Invitrogen, Carlsbad, Calif.) was used for the detection of the bound antibodies. Sup=crude culture supernatant; Tox A=Toxin A; Tox B=Toxin B.

FIG. 4A depicts the effect of pH on the PNPG cleavage activities of toxins A and B. The Cdifftox Activity assay was performed by incubating 100 μg of toxin A or B with 10 mM PNPG at 37° C. for 4 hrs in buffers at the various pH values shown. The following buffers were used for the pH values indicated: glycine-HCl buffer (pH 2-3); citrate buffer (pH 4-6); Tris-HCl buffer (pH 7-10); disodium phosphate-sodium hydroxide buffer (pH 11-12); and KCl—NaOH (pH 13). The assay was monitored by absorbance at 410 nm. Error bars represent standard deviation between two replicate experiments.

FIG. 4B depicts the effect of temperature on the PNPG cleavage activities of toxins A and B. The Cdifftox Activity assay was performed by incubating 100 μg of toxin A or B in 50 mM Tris-HCl containing 50 mM NaCl (pH 7.4) with 10 mM PNPG at the temperatures indicated for 4 hrs. The assay was monitored by absorbance at 410 nm. Error bars represent standard deviation between two replicate experiments.

FIG. 5 depicts a Michaelis-Menten plot for the PNPG cleavage by C. difficile toxins A and B based on non-linear regression method. For toxin A: Km=1.04±0.06 mM and Vmax=1.50±0.03 μmoles/mg/min. For toxin B: Km=0.24±0.02 mM and Vmax=6.40±0.12 μmoles/mg/min. Error bars represent standard deviation from four replicate experiments.

FIG. 6 depicts a dose response inhibition by sodium taurocholate of toxin A and B PNPG cleavage activities. These experiments were performed by incubating for 1 hr 55 μg of each toxin with the amount of sodium taurocholate indicated at 37° C. in 30 mM Tris-HCl buffer (pH 7.4) containing 50 mM NaCl, and 10 mM of the PNPG. Error bars indicate standard deviation from three different experiments.

FIG. 7 depicts a comparison of the Cdifftox Activity assay and ELISA assay for the presence of C. difficile toxins A and B in clinical isolates. Supernatant (250 μl) from isolated strains cultured in BHI media was incubated with 10 mM of PNPG and incubated for 3 hrs at 37° C. The assay was monitored by measuring absorbance at 410 nm. Moles of glucose released was calculated using a molar extinction coefficient for p-nitrophenol of c=17700 M⁻¹cm⁻¹ (53). ATCC strains: 1805=BAA-1805 (tcdA+/B+; NAP1), 057=700057 (tcdA−/B+), 432=43255 (tcdA+/B+), 630=BAA-1382 (tcdA+/B+), Clinical isolates: S1-S14=Culture supernatant from independent clinical isolates obtained from different patients that were tcdA+/tcdB+; C1-C4=Culture supernatant from independent clinical isolates that were tcdA−/tcdB−. Error bars represent the standard deviation between two replicate experiments. Paired t-test analysis showed both ELISA and Cdifftox Activity assay correlated significantly in detecting the presence of the toxins (p=0.001). However, there was not always a correlation between the amount of ELISA signal and the Cdifftox activity. This was expected as the ELISA is not quantitative, whereas the Cdifftox assay is quantitative.

FIG. 8 depicts differentiation of toxigenic and non-toxigenic strains of C. difficile on the Cdifftox Plate assay. Colonies isolated from a stool sample was spread directly onto the plate and incubated anaerobically at 37° C. for 48 hrs. Blue colonies are toxin-producing C. difficile (Tox⁺); pale white colonies are non-toxin producing C. difficile (Tox⁻).

FIG. 9 depicts a schematic representation of the analysis of 50 cytotoxic- and culture-positive stool samples. PCR amplification was performed using the genomic DNA isolated from the Tox⁺ and Tox⁻ colonies to identify a portion of the genes that encode toxin A (tcdA) and toxin B (tcdB).

FIG. 10 depicts results of a PCR analysis of representative Tox⁺ and Tox⁻ strains. Genomic DNA was isolated from the colonies and used as template in PCR reactions with primers specific for the genes that encode toxin A (tcdA) and toxin B (tcdB), and a conserved region of the C. difficile ribosomal RNA (16S rRNA) gene. ‘M’ represents 1 Kb marker (New England BioLabs, Ipswich, Mass.); lanes 1-3: tcdA amplicons; lanes 4-6: tcdB amplicons; lanes 7-9: 16S rRNA amplicons. The PCR products were electrophoresed through a 1% agarose gel and the DNA was detected digitally upon exposure of the ethidium bromide-treated gel to UV light.

FIG. 11 depicts C. difficile toxin production in culture supernatants of a representative Tox⁺ and Tox⁻ clinical isolates and defined ATCC strains. Toxin detection was performed by ELISA and the Cdifftox Activity Assay as described in the examples below. ATCC strains: 1805 represents BAA-1805 strain (tcdA+/B+; NAP1), 432 represents 43255 strain (tcdA+/B+), 630 is historical strain BAA-1382 (tcdA+/B+). Clinical isolates: T1-T14 represents Tox⁺; N1−N6 are Tox⁻ (tcdA− and tcdB−). Error bars represent the standard deviation from two replicate experiments.

### DETAILED DESCRIPTION

The following discussion is directed to various embodiments of the invention. Although one or more of these embodiments may be preferred for some applications, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

### DEFINITIONS

In this disclosure, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements and components comprising one unit and elements or components that comprise more than one unit unless specifically stated otherwise.

The term “indicator-linked substrate” refers to a chromogen or a fluorescent or chemiluminescent molecule that is chemically bound (“linked”) by a glycosidic bond to a sugar moiety on a chemical compound (e.g., glucopyranoside, galactopyranoside) which is recognized by a glycosyltransferase enzyme to cleave the glycosidic bond between the indicator and the sugar moiety. The sugar moiety on the substrate (i.e., chemical compound) may be in either an alpha or beta orientation.

The term “chromogenic substrate” refers to a chromogen-linked substrate in which a chromogen molecule becomes visibly colored or changes color after it is freed or cleaved from the substrate.

As used herein, and unless otherwise indicated, the terms “treat,” “treating,” “treatment” and “therapy” contemplate an action that occurs while a patient is suffering from a C. difficile infection or associated disorder that reduces the severity of one or more symptoms or effects of the C. difficile infection or associated disorder, such as but not limited to bowel or gastrointestinal disorder or a related disease or disorder. Where the context allows, the terms “treat,” “treating,” and “treatment” also refers to actions taken toward ensuring that individuals at increased risk of a C. difficile infection or associated disorder, such as but not limited to bowel or gastrointestinal disorder are able to receive appropriate surgical and/or other medical intervention prior to onset of a C. difficile infection or associated disorder, such as but not limited to bowel or gastrointestinal disorders. As used herein, and unless otherwise indicated, the terms “prevent,” “preventing,” and “prevention” contemplate an action that occurs before a patient begins to suffer from C. difficile infection or associated disorder, such as but not limited to bowel or gastrointestinal disorder, that delays the onset of, and/or inhibits or reduces the severity of, a C. difficile infection or associated disorder, such as but not limited to bowel or gastrointestinal disorder.

As used herein, and unless otherwise indicated, the terms “manage,” “managing,” and “management” encompass preventing, delaying, or reducing the severity of a recurrence of C. difficile infection or associated disorders, such as but not limited to bowel or gastrointestinal disorders in a patient who has already suffered from such a disease, disorder or condition. The terms encompass modulating the threshold, development, and/or duration of the C. difficile infection or associated disorder, such as but not limited to bowel or gastrointestinal disorder or changing how a patient responds to the C. difficile infection or associated disorder, such as but not limited to bowel or gastrointestinal disorder.

As used herein, and unless otherwise specified, a “therapeutically effective amount” of a compound is an amount sufficient to provide any therapeutic benefit in the treatment or management of a C. difficile infection or associated disorder, such as but not limited to bowel or gastrointestinal disorders or to delay or minimize one or more symptoms associated with a C. difficile infection or associated disorder, such as but not limited to bowel or gastrointestinal disorders. A therapeutically effective amount of a compound means an amount of the compound, alone or in combination with one or more other therapies and/or therapeutic agents that provide any therapeutic benefit in the treatment or management of a C. difficile infection or associated disorder, such as but not limited to bowel or gastrointestinal disorders, diarrhea or related diseases or disorders. The term “therapeutically effective amount” can encompass an amount that alleviates a C. difficile infection or associated disorder, such as but not limited to bowel or gastrointestinal disorders, improves or reduces C. difficile infection or associated disorders, such as but not limited to bowel or gastrointestinal disorders, improves overall therapy, or enhances the therapeutic efficacy of another therapeutic agent. By way of example but not limitation, in one embodiment, the therapeutic benefit is inhibiting a bacterial infection or prolonging the survival of a subject with such a bacterial infection beyond that expected in the absence of such treatment.

As used herein, and unless otherwise specified, a “prophylactically effective amount” of a compound is an amount sufficient to prevent or delay the onset of a C. difficile infection or associated disorder, such as but not limited to bowel or gastrointestinal disorders, or one or more symptoms associated with rifamycin sensitive disorders, such as but not limited to bowel or gastrointestinal disorders or prevent or delay its recurrence. A prophylactically effective amount of a compound means an amount of the compound, alone or in combination with one or more other treatment and/or prophylactic agent that provides a prophylactic benefit in the prevention of a C. difficile infection or associated disorder, such as but not limited to bowel or gastrointestinal disorders. The term “prophylactically effective amount” can encompass an amount that prevents C. difficile infection or associated disorder, such as but not limited to bowel or gastrointestinal disorder or a related disease or disorder, improves overall prophylaxis, or enhances the prophylactic efficacy of another prophylactic agent. The “prophylactically effective amount” can be prescribed prior to, for example, travel to a location in which gastrointestinal disorders or diarrhea are common.

As used herein, “patient” or “subject” includes organisms which are capable of suffering from a C. difficile infection or associated disorder, such as but not limited to human and non-human animals. Preferred human animals include human subjects. The term “non-human animals” as used in the present disclosure includes all vertebrates, such as but not limited to, mammals (for example non-human primates, rodents, mice, companion animals and livestock, e.g., sheep, dog, cattle, horses); as well as non-mammals (such as, but not limited to chickens, amphibians, reptiles, etc.).

Susceptible to a C. difficile infection or associated disorder is meant to include, but not be limited to, subjects at risk of developing a C. difficile infection or associated disorder such as but not limited to bowel or gastrointestinal disorders or infections, e.g., subjects suffering from one or more of an immune suppression, subjects that have been exposed to other subjects with a bacterial infection, physicians, nurses, subjects traveling to remote areas known to harbor bacteria, subjects who drink amounts of alcohol that damage the liver, subjects with a history of hepatic dysfunction, etc.

Also, terms such as “element” or “component” encompass both elements and components comprising one unit and elements and components that comprise more than one subunit unless specifically stated otherwise. Also, the use of the term “portion” can include part of a moiety or the entire moiety.

Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, and so forth).

Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, and the like.

### Overview

The presently disclosed methods are based, in part, on the discovery that the A and B toxins of C. difficile cleave chromogenic substrates that have stereochemical characteristics similar to their natural substrate, UDP-glucose. The examples set forth herein demonstrate that methods and compositions for detection of functional C. difficile toxins, and among other things, a quantitative assay (Cdifftox Activity assay) that enables cost-efficient, sensitive, quantitative measurement of the cleavage activities of toxins A and B of C. difficile in a culture supernatant and a selective and differential agar-based assay, the Cdifftox Plate assay (CDPA), which enables identification of toxin-producing C. difficile directly from stool samples without the need for additional toxin-confirmatory tests.

Within the last decade, the incidence of C. difficile infection (CDI) has been increasing, so that it is now the leading definable cause of nosocomial diarrhea. Potential factors that have contributed to this prevalence are the increasing use of intestinal flora-altering antibiotics, the emergence of hypervirulent strains of C. difficile, the propensity of C. difficile to produce recurrent illness refractory to treatment, sub-optimal infection control practice and the appearance of toxin-producing mutant strains with a more potent activity. Pathogenic strains of C. difficile produce either toxin A and/or toxin B, which are important virulent factors in the pathogenesis of this bacterium.

Because of the unmet need, there have been several patent publications directed at C. difficile infection see, for example, WIPO Publication No. WO/1996/040861, entitled “Microbiological Media for Isolation and Identification of Enteric Pathogens such as E. coli and Salmonella” directed to methods and media for the growth, enrichment, isolation, and presumptive identification of enteric pathogens such as E. coli 0157:H7 and Salmonella.

United States Patent Publication No. 20090191583, entitled “Clostridial Toxin Activity Assays” which describes compositions useful for detecting clostridial toxin activity comprising a cell that contains an exogenous clostridial toxin substrate comprises a fluorescent member, a membrane targeting domain and a clostridial toxin recognition sequence comprising a cleavage site, where the cleavage site intervenes between said fluorescent member and said membrane localization domain and methods useful for determining clostridial toxin activity using such clostridial toxin substrates. This method is far more complex than that presently disclosed.

United States Patent Publication No. 20100279330 entitled “Method For Detecting and/or Identifying Clostridium difficile” which relates to a method for detecting and/or identifying Clostridium difficile, characterized in that it comprises the following steps: a) providing a reaction medium comprising at least one beta-glucosidase substrate capable of identifying C. difficile, b) inoculating the medium with a biological sample to be tested, c) allowing for incubation, and d) detecting the hydrolysis of the beta-glucosidase substrate, indicative of the presence of Clostridium difficile. This method identifies and detects C. difficile based on the beta-glucosidase activity of the C. difficile bacteria and would therefore be expected to detect both toxin-producing and non-toxin-producing C. difficile. In contrast, the presently disclosed methods are directed at detecting the functional activities of toxin A and/or toxin B of C. difficile and therefore they selectively identify only pathogenic, toxin-producing strains that cause disease. Furthermore, the presently disclosed methods are based on the glucosyltransferase activity of the C. difficile toxins, not the bacteria. Thus, a non-toxin-producing strain of C. difficile, which does not cause disease, will test negative utilizing the presently disclosed methods but would however appear to be positive in the assay based upon beta-glucosidase activity of the C. difficile bacteria as is described in US20100279330.

In some embodiments are methods of identifying patients infected with C. difficile which are producing functional toxin. Therefore the application of these methods reduces or eliminates the false positive identification of patients suspected of having been infected by a pathogenic strain of C. difficile, for example, based on symptoms. These patients may have symptoms that suggest possible infection with a pathogenic strain of C. difficile, but may actually colonized by C. difficile that is not producing toxin. The initiation of a therapy directed at an infection that the patient does not have, is both a waste of resources and may be unnecessarily detrimental to the patient's health. This results in among other things incorrect identification and incorrect therapy to resolve an infection the patient may not have. Furthermore, such indiscriminate use of antibiotics can lead to drug resistance.

Current clinical methods for diagnosing CDI are mostly qualitative tests that detect either the bacteria or the toxins. The assay described (Cdifftox Activity assay) detect C. difficile toxins A and B activities in a method that is quantitative, cost-efficient, and utilizes a substrate that is stereochemically similar to the native substrate of the toxins, UDP-glucose. The alarming emergence of hypervirulent strains of Clostridium difficile with increased toxin production, severity of disease, morbidity, and mortality emphasizes the need for a culture method that permits simultaneous isolation and detection of virulent strains. C. difficile strains can either be toxin-producing (toxigenic) or non-toxin producing (non-toxigenic). Only toxin A- and/or toxin B-producing strains cause disease. Current culture methods do not differentiate toxigenic and non-toxigenic strains, because they are unable to detect toxin activity. Current methods for diagnosing C. difficile infection are based on detection of the organism, the toxin genes and proteins, or the effect of the cytotoxin on tissue culture cells. The only method that can provide information about the activities of the toxins is the cell cytotoxicity assay. Such limitations are problematic for diagnosis and studies of these toxins. Described in some embodiments is a cost-efficient, sensitive, and reliable assay designated the Cdifftox Activity assay that uses the glucosyltransferase activities of the A and B toxins to identify toxigenic C. difficile. To do this, the Cdifftox Activity assay utilizes PNPG as a chromogenic substrate, which is similar to the native substrate of these toxins.

To characterize toxin activity, toxins A and B were purified from culture supernatants using ammonium sulfate precipitation and chromatography through DEAE-Sepharose and gel filtration columns. The activities of the final fractions were quantitated using the Cdifftox Activity assay and compared to the toxin A- and B-specific ELISA-based antibody assay. The affinity for the substrate was more than 4-fold higher for toxin B than toxin A. Moreover, the rate of cleavage of the substrate was 4.3-fold faster for toxin B than toxin A. The optimum temperature for both toxins ranged between 35-40° C. at pH 8. Culture supernatant from clinical isolates obtained from the stools of patients suspected to be suffering from CDI were tested using the Cdifftox Activity assay and the results were compared to the ELISA assay and PCR amplification of the toxin genes. Our results demonstrate that this new assay is comparable to the current commercial ELISA test for detecting the toxins in the samples tested and has the added advantage of quantitating toxin activity.

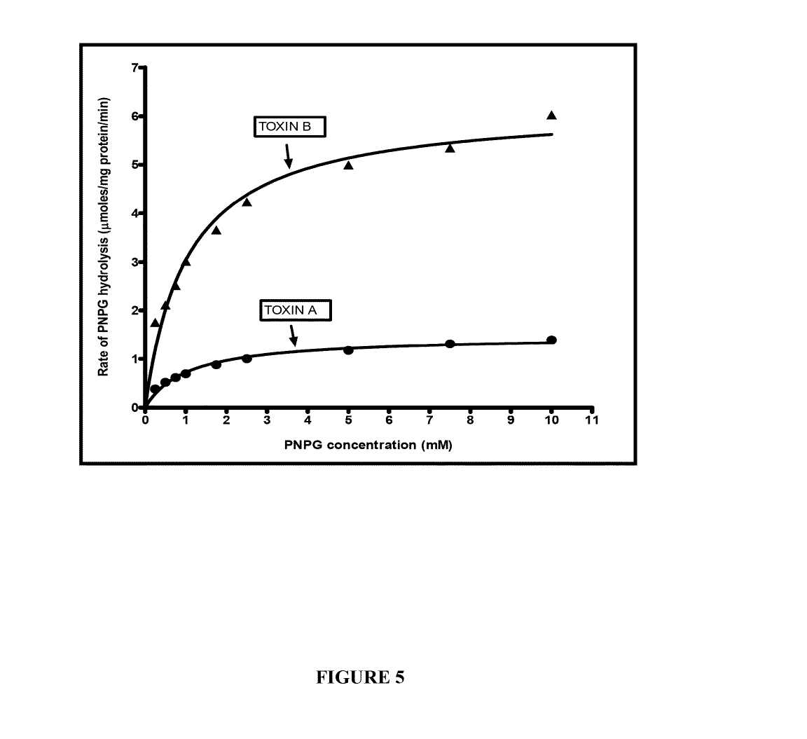

Perhaps as a result, the Michaelis constants (Km) obtained for the toxins with the non-native PNPG substrate (1.04±0.06 mM and 0.24±0.02 mM for toxins A and B, respectively: FIG. 5) are relatively close to those reported for their native UDP-glucose substrate (0.14 mM and 0.18 mM for toxin A and B, respectively). This assay has been used for the purification of C. difficile toxins A and B, and simultaneously evaluated it by comparison to the antibody-based ELISA assay. Unlike commercial ELISA-based assays that only detect the presence of a fragment or region of the toxins, an advantage of the Cdifftox Activity assay is that it detects both presence of the toxins and quantitates their substrate cleavage activities. Thus, the Cdifftox Activity assay identifies the presence of functional toxin.

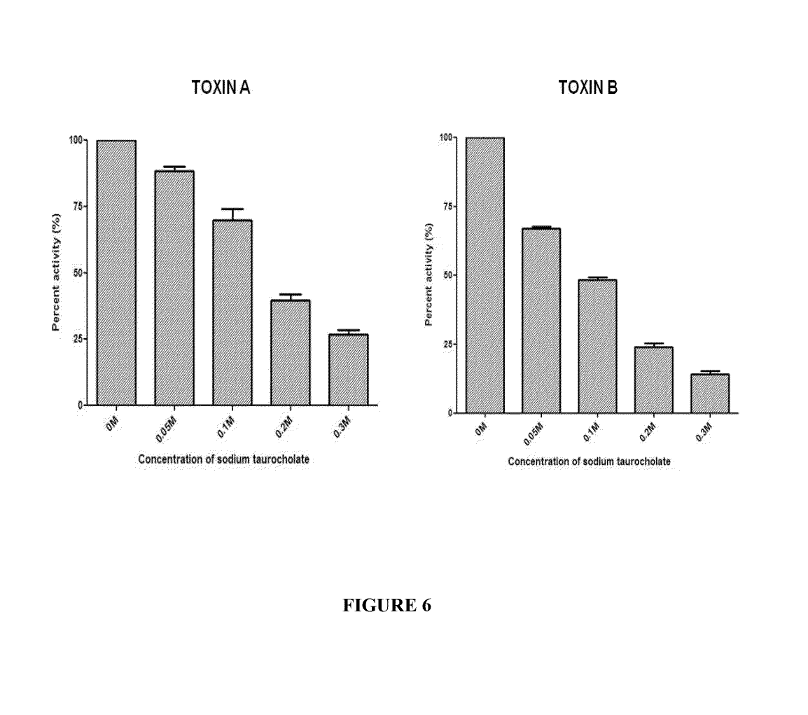

The Cdifftox Activity assay does not distinguish between toxins A and B, since both toxins cleave PNPG and act on the same cellular substrate in vivo. This lack of distinction is of little consequence clinically since both toxins are responsible for the pathogenesis of C. difficile infections. Exemplified below is evidence demonstrating that the PNPG cleavage activities of C. difficile toxins A and B are inhibited by sodium taurocholate in a dose-dependent manner (as seen in FIG. 6). Taurocholate, which is one of the major bile acids found in humans (15, 40) is formed and secreted into the lumen of the small intestine by conjugation of cholic acid with taurine. The total bile acid concentration in the small intestine varies depending on diet and other metabolic conditions. However, only about 2-5% of the bile acids secreted in normal humans enter the colon because the majority of the bile acids are reabsorbed in the ileum. Demonstration of inhibition of the C. difficile toxins by a major bile acid may explain why the pathology of C. difficile infection is almost exclusively restricted to the bile acid-poor colon with relative sparing of the bile-rich small bowel.

Sodium taurocholate is known to non-competitively inhibit mammalian β-glucosidases. These enzymes, including glucosyltransferases, belong to a large family of enzymes that mediate a wide variety of functions such as carbohydrate biosynthesis, metabolite storage, and cellular signaling. Glycosyltransferases transfer a monosaccharide from an activated nucleotide sugar donor to specific sugar residues, proteins, lipids, DNA or small molecule acceptors. This transfer has been shown to occur either by inversion or retention of the configuration of the anomeric carbon. Inhibition of toxin A and B activities by a molecule that also inhibits glucosidases suggest that the cleavage of the PNPG substrate utilized in the Cdifftox Activity assay may be due to the glucosyltransferase activities of the toxins. However, further confirmatory experiments are planned to test the activity of the toxins A and B glucosyltransferase domains. To our knowledge, the use of the glucosyltransferase activities of the A and B toxins to identify toxigenic C. difficile is unique and has not previously been reported.

In some embodiments is an assay (Cdifftox Activity assay) to detect C. difficile toxins A and B activities that is quantitative, cost-efficient, and utilizes a substrate that is stereochemically similar to the native substrate of the toxins, UDP-glucose. Toxin activity was characterized and toxins A and B were purified from culture supernatants using ammonium sulfate precipitation and chromatography through DEAE-Sepharose and gel filtration columns. The activities of the final fractions were quantitated using the Cdifftox Activity assay and compared to the toxin A- and B-specific ELISA-based antibody assay. The affinity for the substrate was more than 4-fold higher for toxin B than toxin A. Moreover, the rate of cleavage of the substrate was 4.3-fold faster for toxin B than toxin A. The optimum temperature for both toxins ranged between 35-40° C. at pH 8. Culture supernatant from clinical isolates obtained from the stools of patients suspected to be suffering from CDI were tested using the Cdifftox Activity assay and the results were compared to the ELISA assay and PCR amplification of the toxin genes. Demonstrating that, among other things, this new assay is comparable to the current commercial ELISA test for detecting the toxins in the samples tested and has the added advantage of quantitating toxin activity.

Strains of C. difficile are broadly classified as either toxin-producing strains (toxigenic) or non-toxin producing strains (non-toxigenic). It has been established that only the toxin-producing strains cause disease and that toxins A and B play critical roles in the pathogenesis of C. difficile. The alarming emergence of hypervirulent strains of C. difficile with increased toxin production, severity of disease, and mortality emphasizes the need for a sensitive diagnostic method that can simultaneously isolate and identify toxigenic strains. Such a method would enable faster and more appropriate treatment of affected patients. The current available culture methods do not differentiate toxigenic and non-toxigenic strains of C. difficile. In some embodiments a Cdifftox Plate assay is described that advances and improves the culture approach by combining the isolation of strains with toxin detection, such that pathogenic toxin-producing strains can be differentiated from non-pathogenic non-toxin-producing strains, which do not cause disease.

The Cdifftox Plate assay (CDPA) identifies toxin-producing C. difficile colonies by their ability to cleave a chromogenic substrate, 5-bromo-4-chloro-3-indolyl-β-D-galactopyranoside, into a distinct insoluble blue product that precipitates around the toxin-producing cells. This substrate cleavage by the toxins was confirmed by the examination of 528 independent C. difficile colonies isolated from 50 stool samples from different patients suffering from C. difficile infection. Although, non-toxigenic strains of C. difficile can also grow on the CDPA plates, none of the non-C. difficile enterobacteriaceae tested could grow on these plates under the same culture conditions. The CDPA plate medium was similarly selective as compared to CCFA (7), in that both culture methods allowed the growth of a similar number of viable colonies from 50 of the 60 stool samples analyzed. Remarkably, 10 stool samples evaluated as positive by the tissue culture cytotoxicity assay did not result in colony growth on any of the culture media utilized (selective and non-selective) under anaerobic conditions. These results suggest that 10 of the 60 cytotoxic-positive samples were false positives. This could have resulted from mishandling of the samples, that the cells in the 10 samples were non-viable, or that the initial cytotoxicity assay results were misinterpreted. This suggests that it is necessary to culture a toxigenic C. difficile bacterium from the stool to confirm diagnosis of an active infection or colonization.

It is important to note that whereas almost all toxin-positive (Tox⁺) colonies (99.8%) on the CDPA plates encoded the toxin genes, 74% of the toxin-negative (Tox⁻) colonies also encoded the tcdA or tcdB genes in their genomes. This suggests that the genomes of some C. difficile strains may encode the toxin genes, but do not secrete detectable amount of toxins. While not wishing to be bound by any particular theory as to any mechanism, it is possible that these proteins are not produced as a consequence of mutations in the toxin-encoding genes. It is also possible that these toxins are not produced due to alternations in the regulatory elements necessary for transcription, translation, or secretion. Alternatively, the bacterial cells may not have been exposed to the necessary conditions to activate toxin gene expression. Factors that have been suggested to influence toxin production are cell density, exposure to antibiotics, phage lysogeny, growth medium composition, and nutrient limitation. Furthermore, C. difficile cells in stool samples may exist as either vegetative cells at different growth stages or as spores. Perhaps, variations in cell physiology explain why some colonies became Tox⁺ later than others. Regardless, the results indicate that this heterogeneity did not lead to false negative interpretations of any of the samples analyzed by the Cdifftox Plate assay.

The present disclosure describes the first use of the glucosyltransferase activities of the A and B toxins to identify toxigenic C. difficile. The Cdifftox Plate assay represents a new detection method with potentially improved sensitivity and efficiency compared to current diagnostic methods. In some embodiments, a selective and differential culture method (Cdifftox Plate assay) combines in a single step the isolation of C. difficile strains with detection of active toxin production. This assay was developed based on our recent finding that the A and B toxins of C. difficile cleave chromogenic substrates that have stereochemical characteristics similar to their natural substrate, UDP-glucose. The Cdifftox Plate assay was validated through the analysis of 528 independent C. difficile isolates selected from 60 tissue culture cytotoxicity-positive clinical stool samples. These isolates were also examined for the presence of the toxin-encoding genes (tcdA and tcdB) in their genomes by PCR amplification. Furthermore, the culture supernatants from the isolates were tested using an enzyme-linked immunosorbent assay for the presence of toxins A and B and the Cdifftox Activity Assay for the presence and activity of toxins A and B. These results demonstrate the Cdifftox Plate assay is 99.8% accurate in detecting toxin-producing C. difficile. The advantage of this new plate-based method is that pathogenic toxin-producing strains can be easily differentiated from non-pathogenic non-toxin-producing strains. As a result, this new method reduces the time and effort required to isolate and confirm toxigenic C. difficile strains.

In some embodiments, a method of identifying the presence of a functional C. difficile toxin, wherein said method comprises: obtaining a sample suspected of containing a C. difficile toxin, adding the sample and a chromogenic substrate with stereochemical characteristics similar to UDP-glucose, incubating the sample and the chromogenic substrate together for a time sufficient to allow the cleavage of the chromogenic substrate in the presence of toxin, determining the amount of cleavage of the chromogenic substrate by monitoring for a change in color by the substrate, or monitoring for a change in absorbance at a predetermined wavelength. For example, in culture media the glucosyltransferase of the C. difficile toxins cleave the chromogenic substrate (e.g., 5-bromo-4-chloro-3-indolyl-β-D-galactopyranoside) into a distinct insoluble blue product that precipitates around the toxin-producing cells or colonies.

In some embodiments the chromogenic substrate is selected from a group consisting of p-nitrophenyl-α-D-glucopyranoside, p-nitrophenyl-β-D-glucopyranoside, 4-aminophenyl-α-D-glucopyranoside, 4-aminophenyl-β-D-glucopyranoside, 5-benzyloxy-3-indoxyl-β-D-glucopyranoside, 5-bromo-6-chloro-3-indoxyl-β-D-glucopyranoside, 6-bromo-2-naphthyl-α-D-glucopyranoside, 6-chloro-3-indoxyl-α-D-glucopyranoside, 6-chloro-3-indoxyl-N-acetyl-beta-D-glucosaminide, 5-bromo-3-indoxyl-β-D-galactopyranoside, and 5-bromo-4-chloro-3-indoxyl-β-D-galactopyranoside. These are representative compounds with stereochemical characteristics similar to UDP-glucose.

In some embodiments the chromogenic substrate is p-nitrophenyl-β-D-glucopyranoside (PNPG). In some embodiments, the chromogenic substrate is in a reagent solution comprising 2-10 mM PNPG, 50 mM Tris-HCl (pH 7.4), 50 mM NaCl, and 100 μM MnCl₂.

In some embodiments, a method of identifying the presence of a functional C. difficile toxin, wherein said method comprises: obtaining a sample suspected of containing a C. difficile toxin, combining 100 μl of the sample and 200 μl of a chromogenic substrate reagent, wherein said reagent comprises 2-10 mM PNPG, 50 mM Tris-HCl (pH 7.4), 50 mM NaCl, and 100 μM MnCl₂, incubating the sample and the chromogenic substrate reagent at 37° C. for 1-4 hrs, stopping the reaction by adding 40 μl of 3 M Na₂CO₃ and determining the cleavage of substrate by measuring the absorbance at 410 nm.

In some embodiments, a method of identifying the presence of a functional C. difficile toxin, for example, a clinical sample wherein said method comprises: obtaining a sample suspected of containing a toxigenic C. difficile, streaking the sample directly onto a plate containing a medium comprising a chromogenic substrate with stereochemical characteristics similar to UDP-glucose, incubating the sample and the chromogenic substrate together for a time sufficient to allow the cleavage of the chromogenic substrate in the presence of toxin, determining the amount of cleavage of the chromogenic substrate by monitoring for a change in color by the substrate. In some embodiments, a method of identifying the presence of a functional C. difficile toxin, in a clinical sample wherein said method comprises: obtaining a sample suspected of containing a toxigenic C. difficile, streaking the sample directly onto a plate comprising a medium that is selective for the growth of C. difficile and comprises a chromogenic substrate with stereochemical characteristics similar to UDP-glucose, incubating the plate in an anaerobic environment for a time sufficient to allow the growth of the C. difficile and the cleavage of the chromogenic substrate in the presence of toxin, determining the amount of cleavage of the chromogenic substrate by monitoring the color of a bacterial colonies.

In some embodiments, a method of identifying the presence of a functional C. difficile toxin, for example, a clinical sample wherein said method comprises: obtaining a sample suspected of containing a toxigenic C. difficile, streaking the sample directly onto a plate comprising a medium that is selective for the growth of C. difficile, and further comprises a chromogenic substrate with stereochemical characteristics similar to UDP-glucose, incubating plate in an anaerobic environment for a time sufficient to allow the growth of C. difficile colonies and the cleavage of the chromogenic substrate in the presence of toxin, determining the presence of functional toxin as a result of cleavage of the chromogenic substrate and monitoring the color of the colonies. In some embodiments, the chromogenic substrate is 5-bromo-4-chloro-3-indolyl-β-D-galactopyranoside. In some embodiments the toxin-producing C. difficile colonies appear blue while non-toxin producers remain pale white. In some embodiments, the medium comprises BHI, peptic digest of animal tissue, pancreatic digest of gelatin, NaCl, dextrose, anhydrous Na₂HPO₄, sodium taurocholate, D-cycloserine and cefoxitin, 5-bromo-4-chloro-3-indolyl-β-D-galactopyranoside, 4-methylphenol, and defibrinated mammal blood such as, but not limited to, defibrinated sheep or horse blood. In some embodiments, the medium comprises BHI broth, sodium taurocholate, D-cycloserine and cefoxitin, 5-bromo-4-chloro-3-indolyl-β-D-galactopyranoside, 4-methylphenol, and defibrinated sheep or horse blood. For example, for some applications, an above-described medium may contain BHI (6 g/L), peptic digest of animal tissue (6 g/L), pancreatic digest of gelatin (14.5 g/L), NaCl (5 g/L), dextrose (3 g/L), anhydrous Na₂HPO₄ (2.5 g/L), sodium taurocholate (0.1%), D-cycloserine (250-500 mg/L) and 8-16 mg/L of cefoxitin, 5-bromo-4-chloro-3-indolyl-β-D-galactopyranoside (100-200 mg/L), 4-methylphenol (0.025%), dimethyl sulfoxide (2-5%) and defibrinated sheep or horse blood (6-8%).

Dimethyl sulfoxide is as an oxidizing agent used to facilitate the enzymatic color reaction even when the cultures remain in an anerobic environment. In addition to dimethyl sulfoxide, other oxidizing agents that can be used include, but are not limited to, those that can support the growth of C. difficile without toxic effects, including ammonium iron (Ill) citrate, ferric ammonium citrate, potassium dichromate and others. The addition of such agents may offer a particular advantage when, for example, the cultures are being maintained outside of the traditional fully equipped clinical laboratory environment, for example, in a anaerobic atmosphere generation bag out in the field facility.

As noted earlier, taurocholate is synthesized in the liver and released into the small bowel. At the ileum, about 95-98% is re-absorbed and channeled into the enterohepatic circulation. Thus, very little, if any taurocholate enters the colon, which is the only part of the body, that C. difficile is known to colonize. Hence, C. difficile releases its potent toxins into the colon that cause the problems associated with its infection. This supports the position that inhibiting the cleavage activity of the C. difficile toxins has a clinical significance. With antibiotic treatment of C. difficile infection becoming increasingly ineffective due to multidrug resistance, inhibiting toxin activity in the colon may be a good approach and sodium taurocholate may be a good candidate for therapy, alone or in combination. Regardless it is clear that the presently disclosed methods and assays may be used to identify the ability of a compound to inhibit the catalytic activity of C. difficile toxin. The presence of therapeutic compounds would inhibit the colorimetric changes associated with C. difficile toxin activity. Therefore, in some embodiments, a method for detecting a compound's ability to inhibit C. difficile toxin activity said method comprising: obtaining a sample suspected of containing a C. difficile toxin, adding the sample and a chromogenic substrate with stereochemical characteristics similar to UDP-glucose, incubating the sample, the compound and the chromogenic substrate together for a time sufficient to allow the cleavage of the chromogenic substrate in the presence of toxin but in the absence of compound, determining the amount of inhibition by the absence of cleavage of the chromogenic substrate by determining a lack of color change in the substrate, or monitoring for a lack of change in absorbance at a predetermined wavelength.

In some embodiments, a method for detecting a compound's ability to inhibit the pathogenesis of C. difficile toxin is identified by the compound's ability to inhibit the toxins cleavage activity, this method comprises: obtaining a sample suspected of containing a C. difficile toxin, adding the sample and a chromogenic substrate with stereochemical characteristics similar to UDP-glucose, incubating the sample, the compound and the chromogenic substrate together for a time known to be sufficient to allow the cleavage of the chromogenic substrate in the presence of toxin, but absence of the compound, determining the amount of inhibition of cleavage of the chromogenic substrate by determining a lack of color change in the substrate, or monitoring for a lack of change in absorbance at a predetermined wavelength.

In some embodiments, a process for identifying a patient infected with toxigenic C. difficile, using the methods described. In some embodiments, a process of identifying an individual as being a candidate for treatment for a toxigenic C. difficile, the process comprising: obtaining a sample or culture from the individual and applying the methods described. In some embodiments, a process of identifying an individual who is infected with toxigenic C. difficile, the process comprising: obtaining a sample or culture from the individual and applying the methods described. In some embodiments, a method of identifying an individual who is need of therapy for a toxigenic C. difficile infection, said method comprising obtaining a sample from the individual and analyzing it using the methods described, for the presence of C. difficile toxins, determining the presence of functional C. difficile toxins and thus identifying the individual as being a valid candidate for therapy.

In some embodiments, a process to identify compounds for use in treating C. difficile toxin mediated disorders, said process comprising the use of the methods disclosed herein. In some embodiments, a test kit for identifying an individual infected with toxigenic C. difficile, comprising the components required to assay a test sample by the methods described. In some embodiments, the described methods are applied to the test samples that are a bodily fluid or cultures obtained from bodily fluids. In some embodiments, the described methods the patient or individual is a mammal selected from the group consisting of human, canine, feline and equine.

### EXAMPLES

### Example 1

### Bacterial Samples and Growth Conditions

Bacterial Strains:

C. difficile toxigenic strains ATCC#s 43255 (tcdA+/B+), the hypervirulent strain BAA-1805 (tcdA+/B+; NAP1), 700057 (tcdA−/B+), and BAA-1382 (tcdA+/B+) were purchased from the American Type Culture Collection (Manassas, Va.). Clinical isolates were obtained from stool samples of hospitalized patients with antibiotic-associated diarrhea suspected to be C. difficile positive (see below). The bacteria were grown in brain heart infusion (BHI)-based medium (Becton Dickinson and Company, Cockeysville, Md.) or on BHI-agar containing cefoxitin (8 μg/ml) and D-cycloserine (300 μg/ml) and liquid or plate cultures were incubated anaerobically in an atmosphere of 10% H₂, 5% CO₂, and 85% N₂ at 37° C. in a Controlled Atmosphere Anaerobic Chamber (PLAS LABS, Lansing, Mich.).

Substrates for Glucosyltransferase:

The substrates 5-bromo-4-chloro-3-indolyl-α-D-glucopyranoside, 5-bromo-4-chloro-3-indolyl-β-D-glucopyranoside, 5-bromo-4-chloro-3-indolyl-α-D-galactopyranoside, 5-bromo-4-chloro-3-indoxyl-β-D-galactopyranoside, 5-bromo-4-chloro-3-indoxyl phosphate, 5-bromo-4-chloro-3-indoxyl butyrate, 5-bromo-4-chloro-3-indoxyl-β-D-xylopyranoside, 5-bromo-4-chloro-3-indoxyl palmitate, 5-bromo-4-chloro-3-indoxyl-α-D-maltotrioside, 5-bromo-4-chloro-3-indoxyl-β-D-glucuronic acid, 5-bromo-4-chloro-3-indoxyl caprylate, and 5-bromo-4-chloro-3-indoxyl choline phosphate, were purchased from Biosynth International (Itasca, Ill.).

These representative chromogenic substrates were selected based on the presence of an O-glycosidic bond between the chromogen and the sugar moiety (glucopyranoside, galactopyranoside, etc.) in either an alpha or beta orientation. They have stereochemical characteristics similar to UDP-glucose, a natural substrate for glucosyltransferase. Other such chromogenic substrates may also be used in the present methods and compositions if desired.

Alternatively, cleavable sugar moieties can also be covalently linked to a fluorescent molecule such as, but not limited to, fluorescein isothiocyanate, rhodamine, phycoerythrin, phycocyanin, allophycocyanin, o-phthaldehyde, and fluorescamine, or a chemiluminescent system such as bioluminescent molecules such as, but not limited to, luciferin, luciferase and aequorin (green fluorescent protein; see, e.g., U.S. Pat. Nos. 5,491,084, 5,625,048, 5,777,079, 5,795,737, 5,804,387, 5,874,304, 5,968,750, 5,976,796, 6,020,192, 6,027,881, 6,054,321, 6,096,865, 6,146,826, 6,172,188 and 6,265,548). Therefore, the use of either a chromogenic or photoluminescent (e.g., fluorescent, chemiluminescent, bioluminescent) substrate for the C. difficile toxin glucosyltransferase enzyme is envisioned for various applications.

Sample Storage Conditions:

The clinical isolates were either stored short-term (less than 1 month) in chopped meat broth (BD Diagnostics, Franklin Lakes, N.J.) at room temperature or long-term in 15% glycerol stocks at −80° C. The purified toxins and eluents were stored at 4° C. for a maximum of one month or until use with no loss of activity. Culture supernatants were stored at 4° C. for a maximum of 2 weeks with no loss of toxin activity.

Clinical Stool Samples:

The clinical stool samples were obtained from an on-going study approved by the Institutional Review Boards of The University of Texas Health Science Center at Houston and St. Luke's Episcopal Hospital (Houston, Tex.). All of the participating patients or their legal guardians provided written informed consent upon admission to the hospital. All the stool samples used were tissue culture cytotoxicity assay-positive, as determined by the Medical Microbiology Laboratory at the St. Luke's Episcopal Hospital.

### Example 2

### Purification of C. difficile Toxins A and B

To purify the toxins, C. difficile strain (ATCC #43255) was cultured anaerobically for 5 days at 37° C. in Spectra/Por dialysis bags (50 ml) with a molecular weight cut-off of 100 kDa (Spectrum Laboratories, Rancho Dominguez, Calif.). Purification of the toxins was performed according to established methods with some modifications. Briefly, the culture was centrifuged at 10,000×g for 10 minutes at 4° C. and the resulting supernatant was filtered through a 0.45 μm membrane filter (Millipore, Billerica, Mass.). To further eliminate low molecular weight proteins, the filtered supernatant was concentrated using a Pierce Concentrator (Thermo Scientific, Rockford, Ill.) with a molecular weight cut-off of 150 kDa. The concentrated supernatant was precipitated by the addition of ammonium sulfate (450 g/L) and incubated overnight at 4° C. with gentle stirring, and subsequently centrifuged at 6,000×g at 4° C. for 20 min. The precipitate was washed and dissolved in 50 mM Tris-HCl buffer (pH 7.4). The sample was loaded onto a fast flow DEAE-Sepharose CL-6B (GE Healthcare Life Sciences, Piscataway, N.J.) anion column pre-equilibrated with buffer D (50 mM Tris-HCl [pH 7.4] containing 50 mM NaCl) at a flow rate of 2 ml/min. The column was washed with buffer D (approximately 350 ml) until all unbound proteins were removed. Toxin A was eluted first with a linear gradient of NaCl (50-250 mM) in buffer D. The elution continued for toxin B with a NaCl gradient of 250-1000 mM in buffer D, after a washing step with 250 ml of buffer D. The fractions (10 ml) were assayed for the presence of toxins by incubating 200 μl with 10 mM PNPG for 3 hr at 37° C. The toxin-positive fractions were pooled and concentrated with 150 kDa Concentrator (ThermoScientific, Pittsburgh, Pa.) for further purification.

The pooled fractions from the DEAE-Sepharose column were further purified by gel filtration chromatography. A 1 cm×100 cm glass Econo column (Bio-Rad Laboratories, Gaithersburg, Md.) was packed with Sephacryl S-300 high resolution beads (GE Healthcare Life Sciences) and calibrated using the following standards purchased from Bio-Rad Laboratories: vitamin B12 (1.35 kDa), myoglobin (17 kDa), ovalbumin (44 kDa), g-globulin (158 kDa), and thyroglobulin (670 kDa). The concentrated toxins were applied to the column and eluted with buffer D at a flow rate of 0.5 ml/min. Fractions (5 ml) were assayed for the presence of the toxins using 200 μl as described above. The purity of the purified toxins was evaluated by electrophoresis through a 5% acrylamide: bisacrylamide PAGE gel (51). The protein concentration of samples was determined using Bradford assay (5) with bovine serum albumin as the standard.

Purification of C. difficile Toxins A and B:

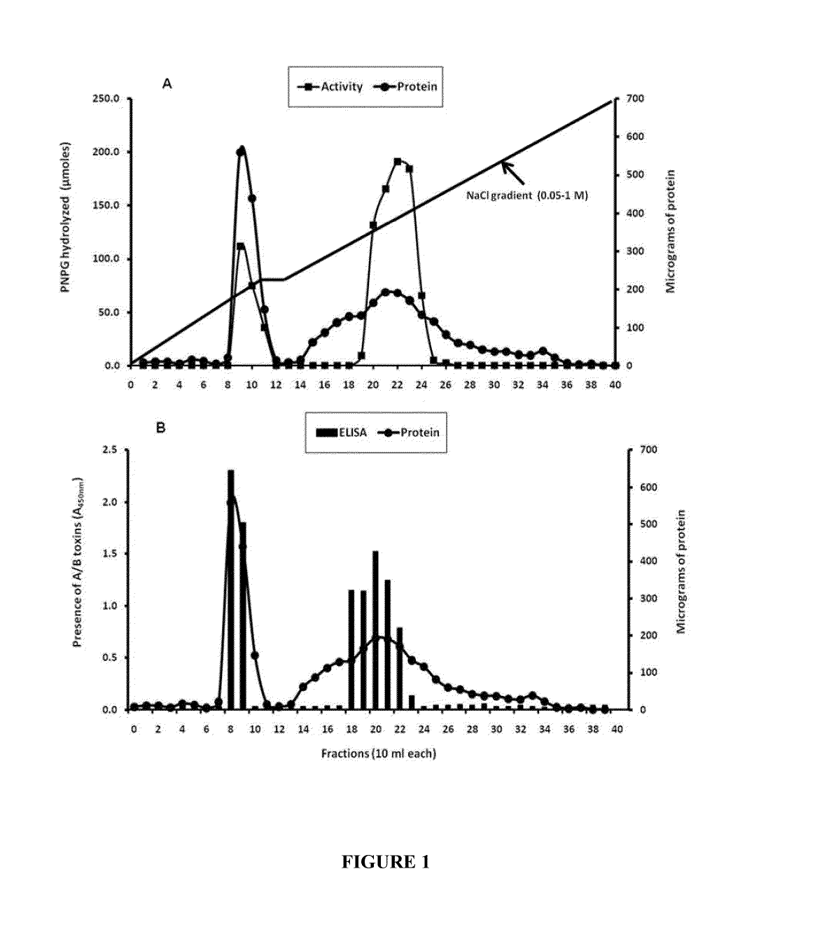

Clostridium difficile toxins A and B were purified seven-fold to characterize and evaluate their substrate cleavage specificities. The native toxins were purified from culture supernatant obtained from the toxin A- and B-positive strain cultured in a dialysis bag of 100 kDa molecular weight cut-off (MWCO). The proteins in the culture supernatant were precipitated with ammonium sulfate, resuspended, and applied to a fast flow DEAE-Sepharose anion exchange chromatography column. After elution with a 50 mM to 1 M NaCl step gradient, two peaks were observed by UV detection and confirmed by Bradford assay (FIG. 1).

The initial narrow peak was determined by sodium dodecyl sulfate polyacrylamide gel electrophoresis (SDS-PAGE) to contain a protein corresponding to the molecular weight of toxin A (308 kDa) and the second broad peak was determined to contain a protein corresponding to toxin B (269 kDa).

Cdifftox Activity Assay:

A Cdifftox Activity assay was used to identify fractions that contained PNPG cleavage activity. The assay consists of the Cdifftox substrate reagent composed of 10 mM PNPG, 50 mM Tris-HCl (pH 7.4), 50 mM NaCl, and 100 μM MnCl₂. The assay was performed in Costar sterile polystyrene 96-well plates (Corning Inc., NY) by adding to each well 200 μl of sample or culture supernatant fluid containing the toxins and 100 μl of the reagent. The plate was incubated at 37° C. for 1-4 hrs and each reaction was stopped by the addition to the well of 40 μl of 3 M Na₂CO₃. Cleavage of the substrate was monitored by measuring the absorbance between 400-500 nm, with a SPECTRA max at 410 nm.

Plus 384 spectrophotometer (Molecular Devices, Sunnyvale, Calif.). To identify the best substrate for the assay, a number of substrates were evaluated including: p-nitrophenyl-α-D-glucopyranoside, 4-aminophenyl-α-D-glucopyranoside, 4-amino-phenyl-β-D-glucopyranoside, 5-benzyloxy-3-indoxyl-β-D-glucopyranoside, 5-bromo-6-chloro-3-indoxyl-β-D-glucopyranoside, 6-bromo-2-naphthyl-α-D-glucopyranoside, 6-chloro-3-indoxyl-α-D-glucopyranoside, 6-chloro-3-indoxyl-N-acetyl-beta-D-glucos-aminide, 5-bromo-3-indoxyl-β-D-galactopyranoside, and 5-bromo-4-chloro-3-indoxyl-β-D-galactopyranoside. PNPG was selected as the substrate of choice because its cleavage by the toxins was the most efficient and sensitive, and had the lowest background. A molar extinction coefficient for p-nitrophenol of ε=17700 M⁻¹cm⁻¹ was used in the calculations (Shikita, M., J. et al., 1999. An unusual case of ‘uncompetitive activation’ by ascorbic acid: purification and kinetic properties of a myrosinase from Raphanus sativus seedlings. Biochem J 341 (Pt 3):725-732). One unit of toxin activity was defined as the amount of the toxins required to cleave one micromole of the PNPG substrate per hour under the experimental conditions. Two toxin-positive fractions were identified that corresponded to the two protein peaks observed (FIG. 1A).

ELISA Assay:

An antibody-based enzyme-linked immunosorbent assay (ELISA), the Wampole C. difficile TOX A/B II assay (TechLab, Blacksburg, Va.) was used as per the protocol provided by the manufacturer to establish the presence of toxins A and B in samples. The presence of toxins A and B in the PNPG active fractions (FIG. 1B) was identified. Specifically, all of the fractions that tested positive using the Cdifftox Activity assay also tested positive using the ELISA assay, and all of the fractions that were negative for the Cdifftox Activity assay were also negative using the ELISA assay. These results indicate that the Cdifftox Activity assay detects the activities of C. difficile toxins A and B.

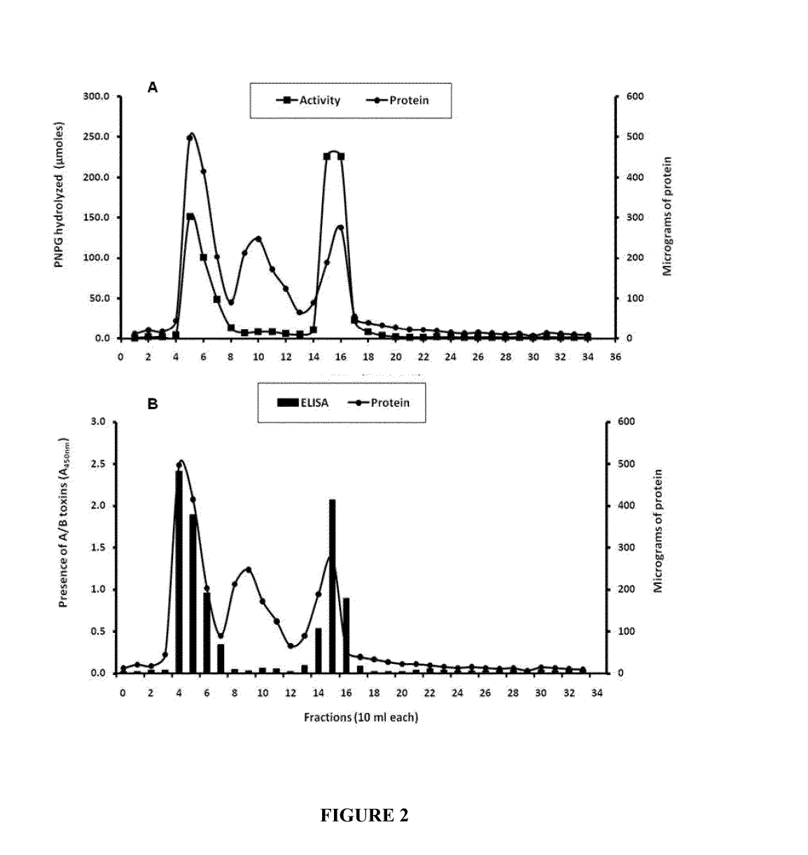

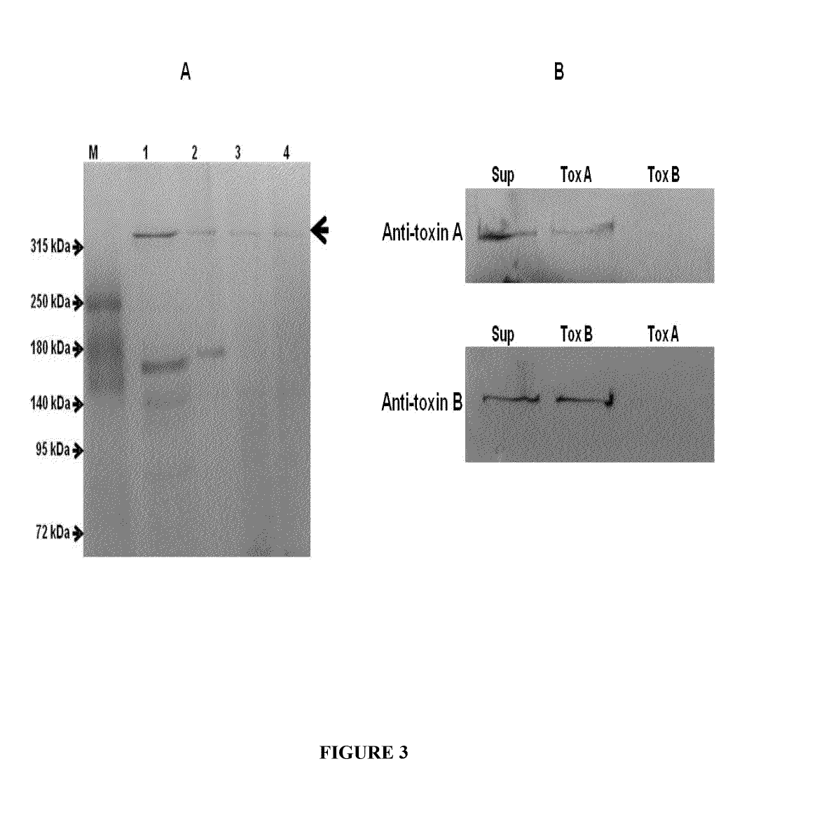

To complete the purification of the C. difficile toxin A and B, the toxin-positive fractions eluted from the DEAE-Sepharose column were pooled, concentrated using a filter with a 150 kDa MWCO, and applied to a Sephacryl S-300 gel filtration column. After elution with buffer D, three predominant peaks were observed by UV detection and confirmed by the Bradford protein assay (FIG. 2A). Examination of the fractions using the Cdifftox assay showed that the PNPG cleavage activity was present in two peaks of different molecular weights. The fractions with PNPG cleavage activities were confirmed by the ELISA assay to contain the toxins (FIG. 2B). Based on the elution profiles of the gel filtration standards used (data not shown), the fractions in the first peak corresponded to toxin A (308 kDa) and those in the second PNPG-active peak corresponded to toxin B (269 kDa). The fractions showing sufficient toxin activity were pooled and concentrated for further analysis by PAGE. The results from the PAGE gel revealed a single visible band in each of the two pooled fractions representing toxins A and B (FIG. 3A). This established that each toxin was purified to homogeneity. The total PNPG substrate cleavage activities of the toxins from each of the purification steps are shown in Table 1. The total enzyme units of cleavage activities of the toxins were enriched by 158-fold. The final substrate cleavage activities of the purified toxins were 0.821 U/mg and 4.7 U/mg for toxins A and B, respectively.

### Example 3

### Characterization of Toxin A and B Activity

To confirm that both toxins A and B cleave the PNPG substrate, Western immunoblot analysis was performed. C. difficile toxins A and B (100 μg each) were separated on 5% PAGE gels. The proteins were transferred from the PAGE gel onto Immun-Blot PVDF membranes (BioRad, Hercules, Calif.) using a Trans-Blot cell (BioRad). The membranes were incubated with individual mouse monoclonal antibodies specific for C. difficile toxins A or B, as the primary antibodies (Abcam, Cambridge, Mass.). The WesternDot 625 Western Blot kit (Invitrogen, Carlsbad, Calif.) was used to probe the membranes for the presence of each toxin. Briefly, the membrane was incubated with Biotin-XX goat anti-mouse IgG secondary antibody and following washing, incubated with the Qdot 625 streptavidin conjugate according to the manufacturer's instructions. Imaging and analysis of the treated membrane was performed using a UVP BioDoc-It Imaging system (Upland, Calif.).

Single bands were observed in each of the samples that had PNPG activity and contained either purified toxin A or B due to their specific reactivity with monoclonal antibodies that recognize toxin A or B, respectively (FIG. 3B). Moreover, toxin A-specific monoclonal antibody did not recognize toxin B, and the antibody specific for toxin B did not recognize toxin A. These results demonstrate that both toxins A and B cleave the PNPG substrate. This is consistent with the reported in vivo activity of these toxins, in that they have both been reported to cleave the same cellular substrate, UDP-glucose.

### Example 4

### The Effect of pH and Temperature on Toxin A and B Activity

The effects of pH and temperature on the functional activities of the toxins were evaluated to determine the optimum temperature and pH for activity. The pH experiments were performed using 5 different buffers to establish a wide range of buffering capacities. For the pH experiment, the following buffers were used: glycine-HCl buffer (pH 2-3); citrate buffer (pH 4-6); Tris-HCl buffer (pH 7-10); disodium phosphate-sodium hydroxide buffer (pH 11-12); and KCl—NaOH (pH 13). Each pH experiment was initiated by incubating 100 μg of toxin A or toxin B with 10 mM of PNPG, followed by incubation in the appropriate buffer at 37° C. for 4 hrs. The reaction was monitored by measuring the absorbance at 410 nm.

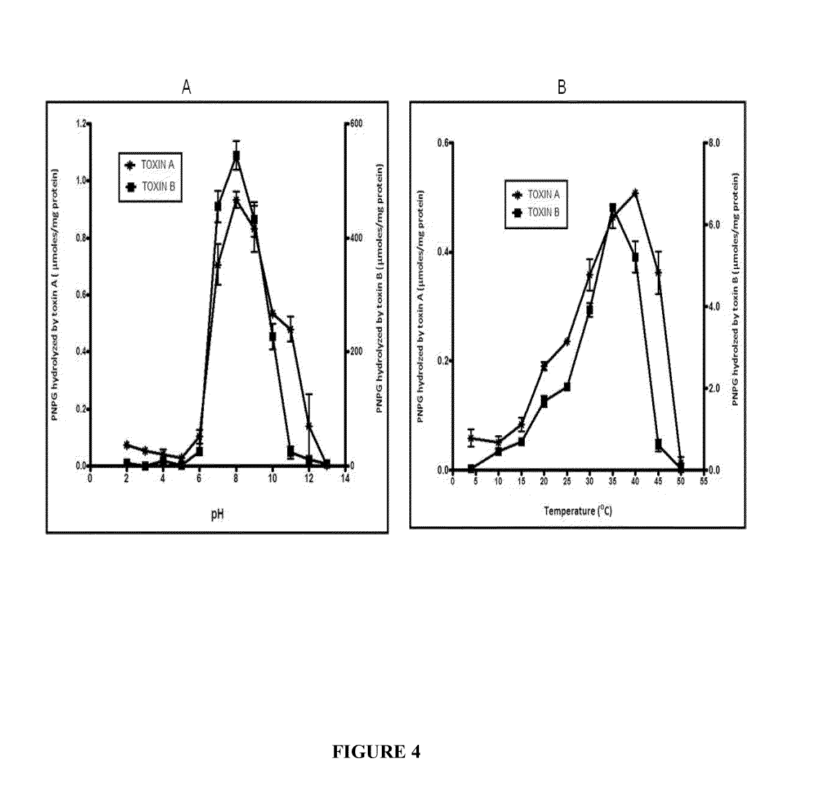

The effect of temperature of incubation on the PNPG cleavage activity was tested in 1.5 ml microcentrifuge tubes using the same conditions as described previously, except that the temperature of incubation was 4, 10, 15, 20, 25, 30, 35, 40, 45, and 50° C. Both toxins A and B demonstrated optimal PNPG cleavage activities within a pH range of 7-9 (FIG. 4). In contrast to toxin A, which showed significant activity within the pH range of 6 to 12, toxin B displayed a more narrow range of PNPG cleavage activity within the pH range of 7 to 10. This is consistent with the pathophysiological environment of the colon, where C. difficile causes disease. The pH of the colon varies from 6.4±0.6 to 7.5±0.4 (Khan, M. S., et al., 2010. Development and eEvaluation of pH-dependent micro beads for colon targeting. Indian J Pharm Sci 72:18-23). Both toxins showed activity optima at a temperature range of 35-40° C., with toxin A showing a broader range of activity than toxin B (FIG. 4).

### Example 5

### Physico-Chemical Analysis of Clostridium difficile Toxins A and B

The amino acid sequences of the toxins were analyzed using the ProtParam program on the ExPASy Proteomics Server (Gasteiger E., et al., 2005. Protein identification and analysis tools on the ExPASy server. John M. Walker (ed): The Proteomics Protocols Handbook, Humana Press) to assess their physico-chemical characteristics. This analysis was performed computationally using the amino acid sequences with accession numbers YP_{—}001087137.1 and YP_{—}001087135.1 (Sebaihia, M., et al., 2006. The multidrug-resistant human pathogen Clostridium difficile has a highly mobile, mosaic genome. Nat Genet 38: 779-786) for toxins A and B, respectively.

Based on the ProtParam analysis, toxin A has 588 total charged residues out of 2710 residues, of which 54% and 46% are negatively and positively charged, respectively. Toxin B has more charged residues (597 out of a total of 2366 residues); 66% and 34% are negatively and positively charged, respectively. These data support the lower isoelectric point (IEP) of 4.42 estimated for toxin B compared to that of toxin A (5.51). The implication of this lower IEP for toxin B is a wide pH range for the maintenance of its overall negative charge at physiological pH. Toxin A is computed to be more stable with an instability index (Guruprasad, K., et al., 1990. Correlation between stability of a protein and its dipeptide composition: a novel approach for predicting in vivo stability of a protein from its primary sequence. Protein Eng 4:155-161) of 29.6 compared to that of 36.5 for toxin B. However, both toxins are estimated to have relatively long in vitro half-lives based on the N-terminal end rule (Tobias, J. W., et al., 1991. The N-end rule in bacteria. Science 254:1374-1377) of 30 hours. These computational data suggest that toxin A should function in and tolerate a wider range of physiological and environmental conditions than toxin B.

### Example 6

### Determination of Km and Vmax

To better define the activities of toxins A and B, a kinetic analysis was performed. A series of experiments were performed to establish the Michaelis-Menten constant (Km) and maximum velocity (Vmax) for the PNPG cleavage activities of each toxin. To determine the amount of the toxins necessary for the experiments, different amounts of each toxin ranging from 30 μg to 120 μg were evaluated with 10 mM of PNPG as substrate. A graph of toxin activity as a function of time was plotted and the amount of each toxin that gave the best linear relationship, but occurred slowly enough for the reaction to be monitored was chosen for the assay. Based on this analysis, 55 μg of toxin A and 100 μg of toxin B were used for the kinetics experiments. Each experiment was repeated four times and the average used for the analysis. The affinities and enzymatic cleavage abilities of each toxin for the PNPG substrate was assessed. Initially, to determine the amount of each toxin that cleaves the substrate at a measurable rate under the experimental conditions, different amounts of the toxins were evaluated at constant substrate concentration. As expected, this resulted in a dose-dependent cleavage of the substrate with increasing toxin amounts. Increasing substrate concentrations also led to an increase in cleavage products as the incubation time increased. The activity of both toxins could be fit to the Michaelis-Menten curve, indicating a single active site reaction (FIG. 5). The Michaelis-Menten constant (Km) values of the toxins for the PNPG substrate were determined by non-linear regression to be 1.04 mM for toxin A and 0.24 mM for toxin B. The maximum velocity (Vmax) for toxin A was 1.5 μmoles/mg/min, whereas that for toxin B was 6.4 μmoles/mg/min. These data indicate that the affinity of toxin B for the PNPG substrate is more than 4-fold higher than toxin A. Moreover, the rate of cleavage of the PNPG substrate is 4.3-fold faster for toxin B than toxin A. These results agree with published assays of the relative damage by toxins A and B to tissue culture cells, in which toxin B was found to be more potent than toxin A.

### Example 7

### C. difficile Activity Inhibition

To further characterize the toxin-substrate interactions, molecules or compounds that could inhibit the activities of toxins A and B were sought. To identify compounds that inhibit the activity of C. difficile toxins A and B, several compounds including sodium taurocholate, dimethyl sulfoxide, phenylmethylsulfonyl fluoride, and dimethyl formamide were tested. Different concentrations of these agents (0, 50, 100, 200 and 300 mM) were added to 55 μg of either toxin A or toxin B in buffer D in a total reaction volume of 300 μl and incubated at 37° C. for 10 minutes. After the toxin-inhibitor incubation period, 10 mM of the PNPG substrate was added and incubated at 37° C. for 1 hr. Absorbance at 410 nm was measured and the percent inhibition was calculated as follows:

$$Percent Inhibition ( \% ) = [ \frac{Specific activity with inhibition}{Specific activity without inhibition} ] \times 100 \%$$

After testing these compounds, sodium taurocholate was observed to inhibit the activities of both toxins. The addition of 300 mM of sodium taurocholate reduced the activities of toxins A and B within one hour of incubation by 71% and 86%, respectively (FIG. 6). Interestingly, taurocholate and phosphatidylserine (both negatively charged lipids) have been reported to inhibit β-glucosidases in a non-competitive manner. These results support the idea that the cleavage of the PNPG substrate is due to the glucosyltransferase/hydrolase activities of the toxins.

### Example 8

### Analysis of Toxin Activity in Clinical C. difficile Isolate Supernatant Fluid

To evaluate the capability of this new Cdifftox Activity assay to detect C. difficile toxins A and B in culture supernatant, C. difficile was isolated from clinical stool samples. Stool samples obtained from patients suspected to be infected by C. difficile were obtained from St. Luke's Hospital (Houston, Tex.) in an IRB-approved study. Single colonies, obtained independently from each patient's stool sample streaked onto BHI-agar media containing cefoxitin (8 μg/ml) and D-cycloserine (300 μg/ml), were inoculated into 10 ml of BHI medium and incubated anaerobically at 37° C. for 72 hrs resulting in an OD_{600 nm} of about 1.3-1.4. After centrifugation at 10,000×g for 10 min at 4° C., 250 μl of the supernatant was incubated with 50 μl of Cdifftox substrate reagent at 37° C. for 3 hours. The assay was quantitated spectrophotometrically at an absorbance of 410 nm. The isolates were not specifically typed to the strain level, but confirmed to be C. difficile based on PCR amplification of the genes that encode the toxins (tcdA and tcdB), as well as toxin production. Culture supernatants from 18 clinical isolates in addition to 4 ATCC strains [BAA-1805 (tcdA+/B+; NAP1), 700057 (tcdA−/B+), 43255 (tcdA+/B+) and BAA-1382 (tcdA+/B+)] were analyzed.

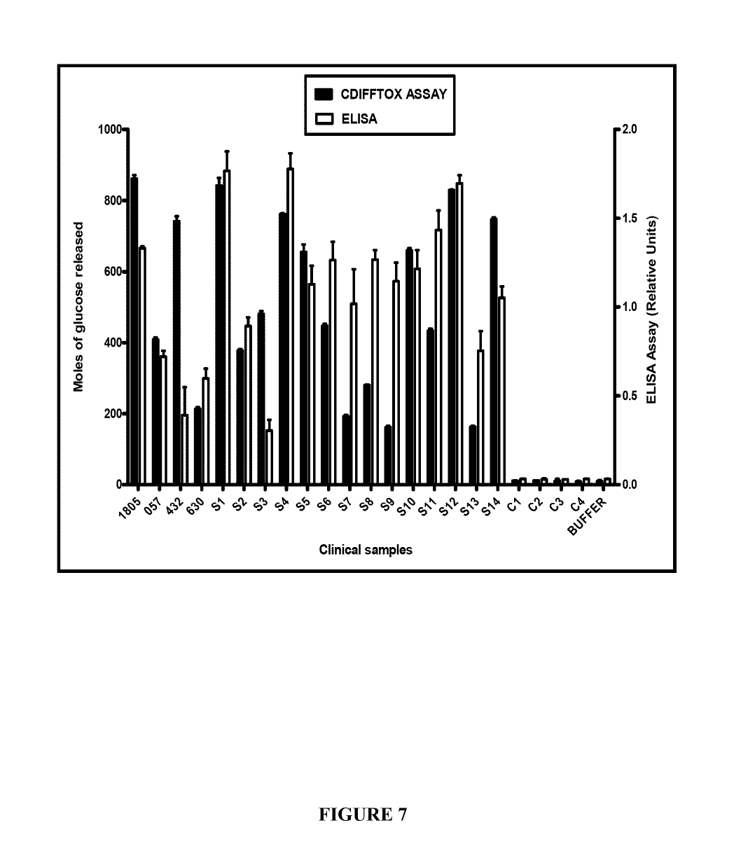

Cultures were prepared from 18 independent clinical isolates from different patients and their supernatants were tested for the presence and activities of toxins A and B using the Cdifftox Activity and ELISA assays (described previously). All the culture supernatants from the clinical isolates determined to be positive for the toxins by the Cdifftox Activity assay were also positive by the ELISA assay (FIG. 7). The toxin-negative culture supernatants were negative in both assays. Genomic DNA was isolated from each strain and PCR amplification analysis was performed with specific primers to identify the genomes encoding the C. difficile tcdA (toxin A) and tcdB (toxin B). The toxin gene-positive isolates matched those that were toxin-positive by the Cdifftox Activity and ELISA assays. Paired t-test analysis showed both ELISA and Cdifftox Activity assay correlated significantly in detecting the presence of the toxins (p=0.001). However, there was not always a correlation between the amount of ELISA signal and the Cdifftox activity. This was expected as the ELISA is not quantitative, whereas the Cdifftox assay is quantitative.

Polymerase Chain Reaction Amplification of C. difficile Toxin Genes:

The presence of the toxin genes (tcdA and tcdB) and the 16S rRNA gene in the clinical isolates was confirmed by PCR amplification. Genomic DNA was isolated from 1 ml of culture at an optical density of 0.75 at 600 nm using the DNAEasy kit (Qiagen, Valencia, Calif.). Amplification was performed using Phire Hot Start DNA Polymerase (Finnzymes, Woburn, Mass.). The following primers were used: toxin A (Forward-5′TGATGCTAATAATGAATCTAAAATGGTAAC3′ (SEQ ID NO: 1) and Reverse-5′ ACCACCAGCTGCAGCCATA3′ (SEQ ID NO: 2)); toxin B (Forward-5′GTGTAGCAATGAAAGTCCAAGTTTACGC3′ (SEQ ID NO: 3) and Reverse-5′CACTTAGCTCTTTGATTGCTGCACCT3′ (SEQ ID NO: 4)) and 16S rRNA (Forward-5′ ACACGGTCCAAACTCCTACG3′ (SEQ ID NO: 5) and Reverse-5′ AGGCGAGTTTCAGCCTACAA3′ (SEQ ID NO: 6)). The DNA was amplified with an initial denaturation of 98° C. for 30 sec and 36 cycles of 98° C. for 10 sec, 62° C. for 10 sec and 72° C. for 10 sec with a final extension of 72° C. for 1 min. The PCR products were analyzed using 1.5% agarose gel electrophoresis.

Interestingly, it was determined that some of the isolates that were confirmed by PCR to encode tcdA and tcdB and tested positive with the Cdifftox Activity assay, had initially tested negative using the ELISA assay. However, these isolates became ELISA positive following increased incubation of the culture, suggesting that the Cdifftox Activity assay is more sensitive than the ELISA assay. These findings illustrate that the Cdifftox Activity assay is a sensitive and reliable method to detect and assess the functional activities of C. difficile toxins A and B in culture supernatant.

### Example 9

### Cdifftox Plate Assay

The Cdifftox Plate assay uses a novel selective and differential agar-based culture medium to specifically allow the growth of C. difficile and simultaneously identify colonies producing active toxins A and B, while inhibiting the growth of non-C. difficile colonies.

Cdifftox Plate Assay Medium:

This agar-based culture medium was developed to specifically allow the growth of C. difficile and simultaneously identify toxins A- and B-producing colonies, while inhibiting the growth of non-C. difficile colonies. To identify a substrate that is stereochemically identical to UDP-glucose, the native substrate of the C. difficile toxins A and B, all the chromogenic substrates listed above were evaluated for cleavability and the stability of the product. After testing different compositions of potential substrates and various compounds, the following components were chosen to compose this new Cdifftox Plate assay (CDPA) medium (per liter): BHI (6 g), peptic digest of animal tissue (6 g), pancreatic digest of gelatin (14.5 g), NaCl (5 g), dextrose (3 g), anhydrous Na₂HPO₄ (2.5 g), sodium taurocholate (0.1%) (Sigma-Aldrich, St. Louis, Mo.), D-cycloserine (250-500 mg) and 8-16 mg of cefoxitin (Fisher Scientific, Pittsburgh, Pa.), 5-bromo-4-chloro-3-indolyl-β-D-galactopyranoside (100-200 mg), 4-methylphenol (0.025%), agar (12-14 g), and defibrinated sheep or horse blood (6-8%). Alternatively, the CDPA medium can also be prepared with 37 g of BBL BHI broth, sodium taurocholate (0.1%) (Sigma-Aldrich, St. Louis, Mo.), D-cycloserine (250-500 mg) and 8-16 mg of cefoxitin (Fisher Scientific, Pittsburgh, Pa.), 5-bromo-4-chloro-3-indolyl-β-D-galactopyranoside (100-200 mg), 4-methylphenol (0.025%), agar (12-14 g), and defibrinated sheep or horse blood (6-8%). Prior to application of the samples, the CDPA plates were placed in the anaerobic chamber for 4 hrs. Dimethly sulfoxide can be used to facilitate color development of the substrate cleavage product under anaerobic environment. However, color development can also be achieved by incubating the CDPA plates aerobically for at least 30 minutes.

It was determined that a low level of sodium taurocholate (0.1% or approximately 1.9 mM) serves as a germinant in the plate assay medium to enable the C. difficile spores that present in stool to germinate into vegetative cells. At this low concentration, it does not inhibit the cleavage activity of the toxins. The CDPA still works without the taurocholate but more C. difficile colonies are consistently observed in the presence of taurocholate.

Cdifftox Plate Assay:

For the Cdifftox Plate assay, each stool sample was streaked directly onto two CDPA plates using a sterile loop. The plates were incubated anaerobically for 24-72 hrs at 37° C., until colonies appeared. The presumptive toxin-producing C. difficile colonies appeared blue while non-toxin producers remained pale white. The blue colonies were phenotypically classified as Tox⁺ (presumably tcdA⁺ and/or tcdB⁺), whereas the pale white colonies were denoted Tox⁻ (presumably tcdA⁻ and tcdB⁻ or mutants with genetic alterations that affect toxin production or activity).

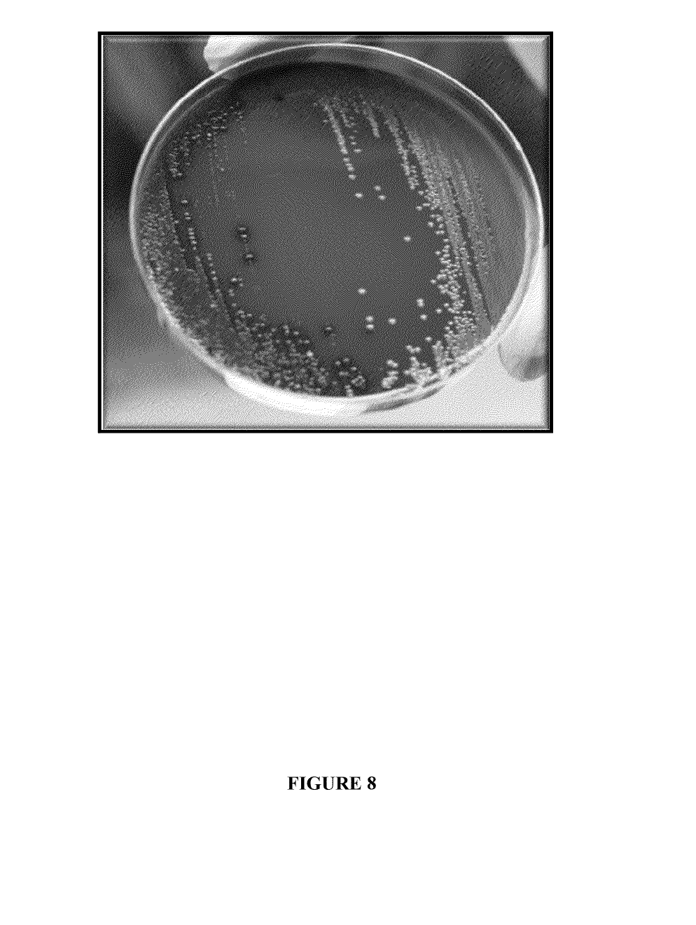

The assay was initially tested using the well-characterized toxigenic C. difficile strains ATCC 43255 (tcdA+/B+), ATCC BAA-1382 (tcdA+/B+), ATCC 700057 (tcdA−/B+), and the hypervirulent strain, ATCC BAA1805 (tcdA+/B+). After 24 hrs of incubation, colonies of these strains that were producing high levels of the toxins appeared blue (Tox⁺), whereas those that were producing less toxins remained pale white (Tox⁻), similar to the colonies shown in FIG. 8. By 48 hrs, all the colonies had turned blue, indicating they were producing active toxins.

### Example 10

### Characterization of Toxigenic C. difficile Using the Cdifftox Plate Assay

To evaluate the detection of toxigenic strains of C. difficile from clinical stool samples using this new assay, 60 tissue culture cytotoxicity assay-positive clinical stool samples collected at the St. Luke's Episcopal Hospital (Houston, Tex.) were tested. The stool samples were spread directly onto the CDPA plates and incubated anaerobically at 37° C. for 24-72 hrs. Viable bacterial colonies were successfully isolated from 50 of the 60 stool samples analyzed. The proportion of Tox⁺ to Tox⁻ colonies from the 50 stool samples that grew on the CDPA plates after 48 hrs of incubation was as follows: 23 samples produced 100% Tox⁺ colonies, 14 samples produced approximately 60-85% Tox⁺ colonies, 4 samples produced 30-50% Tox⁺ colonies, whereas 1 sample produced 10% Tox⁺ colonies. Interestingly, the proportion of Tox⁺ colonies increased as the incubation time was increased to a maximum of 72 hrs; some of the colonies that were initially Tox⁻ became Tox⁺ with increasing incubation period, however, none of the Tox⁺ became Tox⁻. All 42 colonies that were Tox⁻ at 72 hrs remained Tox⁻, even after transfer to a new plate.

The selectivity of the CDPA medium for C. difficile strains was compared with BHI-agar, a non-selective medium and another C. difficile selective medium Closerine-Cefoxitin Fructose Agar (CCFA) medium, which was prepared as follows per liter: proteose peptone #2 (40 g), anhydrous Na₂HPO₄ (5 g), anhydrous KH₂PO₄ (1 g), NaCl (2 g), anhydrous MgSO₄ (0.1 g), fructose (6 g), neutral red (0.003%), D-cycloserine (500 mg), cefoxitin (15.5 mg), and agar (15 g). To test for the growth of other anaerobes present in the stool, the samples were also cultured on BHI-agar plates without antibiotics. All plates were incubated and grown for 1-3 days under anaerobic conditions at 37° C. All 60 stool samples were spread directly onto each plate and incubated anaerobically at 37° C. for 24-72 hrs. The CCFA and BHI-agar media allowed the growth of colonies from the same 50 of the 60 stool samples tested as the CDPA medium. Overall, more bacterial colonies were observed on the CCFA (approximately 15%) and BHI-agar media (about 40%) compared to the CDPA medium.

To demonstrate the specificity of the Cdifftox Plate assay, non-Clostridium difficile bacteria were tested for growth under the same culture conditions as the clinical stool samples. The growth of non-Clostridium difficile bacteria on the CDPA medium under the standard conditions used for the stool samples was examined. The following strains were tested: Bacteroides fragilis, B. thetaiotaomicron, Campylobacter jejuni, C. perfringens, Enterobacter cloacae, enteropathogenic Escherichia coli, enterotoxigenic E. coli H10407, Lactobacillus spp, Plesiomonas shigelloides, Salmonella enteritica, Shigella flexneri, Staphylococcus aureus, Vibrio alginolyticus, V. parahaemolyticus, and Yersinia enterocolitica.

No viable colonies were observed when pure cultures of Bacteroides fragilis, B. thetaiotaomicron, Campylobacter jejuni, C. perfringens, Enterobacter cloacae, enteropathogenic Escherichia coli, enterotoxigenic E. coli H10407, Lactobacillus spp, Plesiomonas shigelloides, Salmonella enteritica, Shigella flexneri, Staphylococcus aureus, Vibrio alginolyticus, V. parahaemolyticus, and Yersinia enterocolitica were streaked on the CDPA plates.

These findings suggest that the Cdifftox Plate assay discriminates non-C. difficile bacteria with a selectivity that is comparable to that of CCFA media in isolating viable C. difficile colonies directly from stool.

### Example 11

### PCR Amplification of C. difficile Toxin-Encoding Genes

A total of 528 single colonies consisting of 486 Tox⁺ and 42 Tox⁻ colonies were selected from the CDPA plates for further analysis. A total of 10-12 independent isolates were selected from each stool sample; when possible both Tox⁺ and Tox⁻ colonies were selected from each sample. The presence of the toxin-encoding genes (tcdA or tcdB) in the genomes of the presumptive Tox⁺ and Tox⁻ isolates was examined by PCR amplification of a portion of each of these genes. A portion of the 16S ribosomal RNA (rRNA) genes was also amplified. These reactions were performed by first isolating genomic DNA from 1 ml of an overnight culture of each isolated colony using the DNeasy Blood and Tissue Kit (Qiagen, Germantown, Md.). Amplification was performed using Phire Hot Start DNA Polymerase 11 kit (Finnzymes, Woburn, Mass.). The previously described forward and reverse primers were used for toxin A (SEQ ID NOS: 1 and 2), toxin B (SEQ ID NOS: 3 and 4) and 16S rRNA (SEQ ID NOS: 5 and 6). The DNA was amplified with an initial denaturation of 98° C. for 30 sec and 36 cycles of 98° C. for 10 seconds, 60° C. for 10 sec and 72° C. for 10 sec, with a final extension of 72° C. for 1 min.

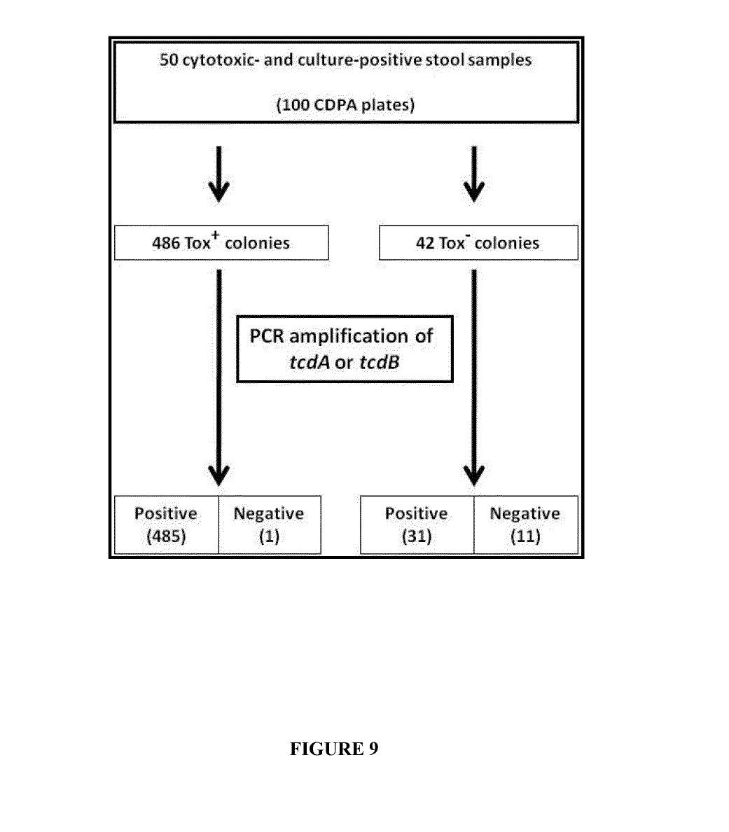

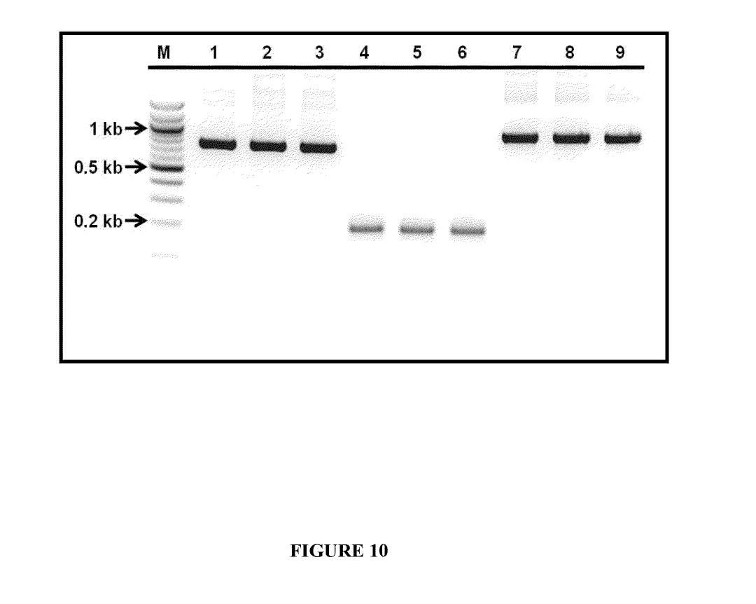

To confirm that the Tox⁺ colonies isolated using the Cdifftox Plate assay possessed the genes in their genomes that encode the toxins, a total of 528 single bacterial colonies comprised of 486 Tox⁺ and 42 Tox⁻ independent clinical isolates from the 50 stool samples were examined by PCR amplification (FIG. 9). Genomic DNA was purified from the selected colonies and used as a template for the amplification of an 800 bp portion of the conserved region of the C. difficile 16S rRNA gene, and a portion of the genes that encode toxin A (tcdA) and toxin B (tcdB). All the 528 total isolates tested were positive for the conserved region of the C. difficile 16S rRNA gene (FIG. 10). Of the 486 Tox⁺ isolates evaluated, 485 (99.8%) were positive for either tcdA and/or tcdB (FIG. 9). Of the 42 Tox⁻ isolates evaluated, 31 (74%) were positive for either tcdA and/or tcdB, whereas 11 (26%) were negative for both tcdA and tcdB. These data indicated that 100% of the genomes of the Tox⁺ and Tox⁻ isolates that were selected on the CDPA plates from the stool samples were C. difficile. Furthermore, 100% of the Tox⁺ strains encode the genes for synthesis of either C. difficile toxin A and/or toxin B. Interestingly, 74% of the Tox⁻ strains encoded one or both of the toxin genes in their genomes.

### Example 12

### Toxin Assays

Single colonies (486 Tox⁺ and 42 Tox⁻) were inoculated into 10 ml of BHI medium and incubated anaerobically at 37° C. for 72 hrs resulting in an OD_{600 nm} of about 1.3-1.4. After centrifugation at 10,000×g for 10 min to remove the cells, the culture supernatants were collected and stored at 4° C. until use.

The presence of toxins A and/or B in the culture supernatants from the isolates was evaluated using the Wampole C. difficile TOX A/B II (TechLab, Blacksburg, Va.). This assay was performed according to the protocol provided by the manufacturer.

Cdifftox Activity Assay:

The activity of toxins A- and B-producing C. difficile isolates was quantitated using the Cdifftox Activity assay described previously in Example 2. The assay was performed on 250 μl of each sample supernatant fluid to which 100 μl of the substrate reagent (10 mM p-nitrophenyl-β-D-glucopyranoside, 50 mM Tris-HCl, (pH 7.4), 50 mM NaCl, and 100 μM MnCl₂) was added in a Costar sterile polystyrene 96-well plate (Corning Inc., NY). Each reaction was incubated at 37° C. for 2-4 hours and stopped by the addition of 40 μl of 3 M Na₂CO₃. Cleavage of the substrate was monitored by absorbance measurement at 410 nm using a SPECTRA max Plus 384 spectrophotometer (Molecular Devices, Sunnyvale, Calif.). A molar extinction coefficient for p-nitrophenol of ε=17700 M⁻¹ cm⁻¹ was used in the calculations (53).

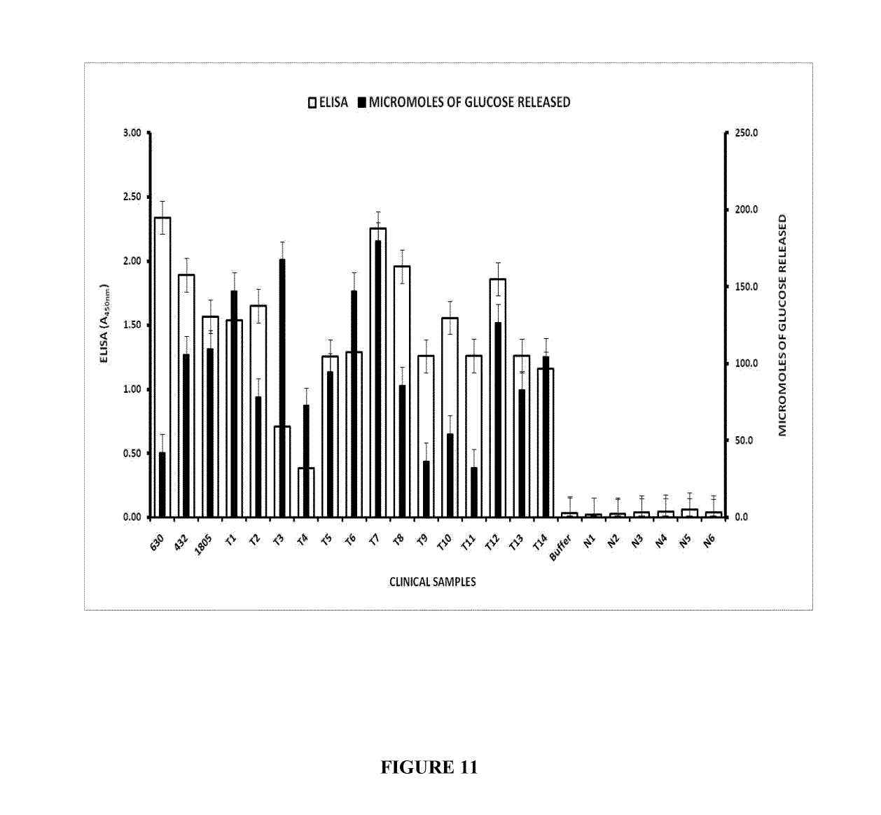

The Cdifftox Plate assay differentiates toxigenic from non-toxigenic C. difficile colonies via the activities of the toxins (either toxin A or B). To ensure that the Tox⁺C. difficile cells were able to secrete active toxins, the presence and activity of toxins in the culture supernatants of Tox⁺ and Tox⁻ isolates were evaluated. Toxin detection was performed on culture supernatants from three of each stool sample by ELISA, an antibody-based assay, commonly used in clinical laboratories. Toxin detection and activity was tested of all the isolates by the Cdifftox Activity assay, described previously in Example 2. All the culture supernatants from the clinical isolates that were positive for tcdA and/or tcdB by PCR amplification and tested by ELISA (150 Tox⁺) and Cdifftox Activity assays (485 Tox⁺) were positive for the presence and activity respectively, of the toxins (FIG. 11). Remarkably, all the colonies determined by the Cdifftox Plate assay to be Tox⁻, whether they encoded tcdA or tcdB in their genomes or not, were negative for the presence and toxin activity in both of these assays. These results confirmed that all but one of the 486 Tox⁺ colonies selected on the Cdifftox Plate assay were toxin-producing (toxigenic) C. difficile. Thus, the Cdifftox Plate assay specifically and reliably detects toxigenic C. difficile in clinical stool samples. In contrast to other culture media available for isolating C. difficile, the Cdifftox Plate assay is advantageous in that it combines selective growth of the bacteria with the detection of the active toxins in a single step. Thus, drastically reducing the time and effort required to isolate and confirm an infection resulting from toxigenic C. difficile strains.

Data Analysis:

All the data were analyzed and plotted using GraphPad Prism version 5.02 for Windows (GraphPad Software, San Diego, Calif.). The nonlinear regression method was used to calculate the Km and Vmax values. Paired t-test was used to compare the performance of the new Cdifftox Activity assay in detecting the presence of the toxins in comparison with ELISA. In all cases, statistical significance was defined as p<0.05.

### Example 13

### Test Kits

In some embodiments, identifying a patient infected with toxigenic C. difficile is done using a kit that contains all of the reagents required. All the materials and reagents required for conducting such determinations may be assembled together in a kit to facilitate the rapid and easy identification of patient samples containing toxigenic C. difficile using the methods and reagents described in the examples above, such as but not limited to those of the Cdifftox Activity Assay and Cdifftox Plate Assay. With the kit, the user determines whether a particular sample of a patient's bodily fluid or a subculture of the sample contains functional C. difficile toxin (i.e., glucosyltransferase activity). These exemplary assays may be performed as two distinct assays or in combination or confirmatory of each another.

In some embodiments, the kit contains the necessary components for testing a bodily fluid for active C. difficile toxin, to determine whether the individual is infected with toxigenic C. difficile and in need of therapy. A test kit may have a single container or it may include individual containers for each reagent. When test components are provided in the form of one or more liquid solutions, they are preferably provided as sterile aqueous solutions. Reagents may also be provided in dried or lyophilized forms. When reagents or components are provided as a dried form, reconstitution generally is by the addition of a suitable solvent. The solvent may be provided in another container means. The kit may also include one or more vials, test tubes, flasks, bottles, syringes or other suitable containers, into which the test reagent formulation is placed, preferably suitably allocated. In some embodiments, a kit also comprises a second container for containing a sterile, pharmaceutically acceptable buffer or other diluent. For example, the container may itself be a syringe, pipette, or other dispensing device that can be used for applying or mixing with other components of the kit. Irrespective of the number or type of containers employed in a kit, in some embodiments the kit also includes, or is packaged with, an instrument for signal detection and analysis. In some embodiments, a kit includes suitable packaging for holding the various components (e.g., vials) in close confinement for commercial sale such as, for example, an injection or blow-molded plastic container in which the desired vials are retained. Instructions for use of the kit components may be provided in the kit.

Lateral flow tests or test strips, are a logical expression of the described methods to facilitate assessment of C. difficile toxin levels. The principle behind the test strip is straightforward: the cleavable chromogenic substrate is bound to a solid support in such a way as to permit the C. difficile toxin to cleave it. The potential benefits of test strips in some cases include their user-friendly format, short time to provide test result, long-term stability over a wide range of climates, and relatively inexpensive production cost. These features make strip tests ideal for many applications such as home testing, rapid point of care testing, and testing in the field and during transport to a medical facility, or rapidly upon arrival at such a facility. In addition, test strips may provide reliable testing that might not otherwise be available to rural environments or third world countries.

### REFERENCES

The following references may be helpful for understanding the invention. To the extent that they provide procedural or other details supplementary to those set forth herein, they are specifically incorporated herein by reference.
E. Fernandez-Salas, et al., U.S. Pat. App. Pub. No. 20090191583, entitled “Clostridial Toxin Activity Assays”, dated Jul. 30, 2009.
K. Asir, et al., U.S. Pat. App. Pub. No. 20100279330 entitled “Method For Detecting and/or Identifying Clostridium difficile”, dated Nov. 4, 2010.
B. Bochner, WIPO Int. Pat. Pub. No. WO/1996/040861, entitled “Microbiological Media for Isolation And Identification of Enteric Pathogens Such as E. coli and Salmonella,” dated Dec. 19, 1996.
1. Bachmair, A., D. Finley, and A. Varshaysky. 1986. In vivo half-life of a protein is a function of its amino-terminal residue. Science 234:179-186.
2. Barbut, F., R. Revel, R. Ephraim, P. Leluan, P. Lureau, and J. C. Petit. 1994.
Evaluation of a rapid enzyme immunoassay for the detection of Clostridium difficile in stools. Eur J Clin Microbiol Infect Dis 13:277-278.
3. Bartlett, J. G. 1992. Antibiotic-associated diarrhea. Clin Infect Dis 15:573-581.
4. Bartlett, J. G. 2002. Clinical practice. Antibiotic-associated diarrhea. N Engl J Med 346:334-339.
5. Bradford, M. M. 1976. A rapid and sensitive method for the quantitation of microgram quantities of protein utilizing the principle of protein-dye binding. Anal Biochem 72:248-254.
6. Breton, C., L. Snajdrova, C. Jeanneau, J. Koca, and A. Imberty. 2006. Structures and mechanisms of glycosyltransferases. Glycobiology 16:29R-37R.
7. Campbell, J. A., G. J. Davies, V. V. Bulone, and B. Henrissat. 1998. A classification of nucleotide-diphospho-sugar glycosyltransferases based on amino acid sequence similarities. Biochem J 329 (Pt 3):719.
8. Choy, F. Y., and R. G. Davidson. 1980. Gaucher's disease II. Studies on the kinetics of beta-glucosidase and the effects of sodium taurocholate in normal and Gaucher tissues. Pediatr Res 14:54-59.
9. Ciesla, W. P., Jr., and D. A. Bobak. 1998. Clostridium difficile toxins A and B are cation-dependent UDP-glucose hydrolases with differing catalytic activities. J Biol Chem 273:16021-16026.
10. Delmee, M. 2001. Laboratory diagnosis of Clostridium difficile disease. Clin Microbiol Infect 7:411-416.
11. Delmee, M., T. Mackey, and A. Hamitou. 1992. Evaluation of a new commercial Clostridium difficile toxin A enzyme immunoassay using diarrhoeal stools. Eur J Clin Microbiol Infect Dis 11:246-249.
12. Depitre, C., V. Avesani, M. Delmee, and G. Corthier. 1993. Detection of Clostridium difficile toxins in stools. Comparison between a new enzyme immunoassay for toxin A and other routine tests. Gastroenterol Clin Biol 17:283-286.
13. Dillon, S. T., E. J. Rubin, M. Yakubovich, C. Pothoulakis, J. T. LaMont, L. A. Feig, and R. J. Gilbert. 1995. Involvement of Ras-related Rho proteins in the mechanisms of action of Clostridium difficile toxin A and toxin B. Infect Immun 63:1421-1426.
14. Dingle, T., S. Wee, G. L. Mulvey, A. Greco, E. N. Kitova, J. Sun, S. Lin, J. S. Klassen, M. M. Palcic, K. K. Ng, and G. D. Armstrong. 2008. Functional properties of the carboxy-terminal host cell-binding domains of the two toxins, TcdA and TcdB, expressed by Clostridium difficile. Glycobiology 18:698-706.
15. Dowling, R. H. 1973. The enterohepatic circulation of bile acids as they relate to lipid disorders. J Clin Pathol Suppl (Assoc Clin Pathol) 5:59-67.
16. Egerer, M., T. Giesemann, T. Jank, K. J. Satchell, and K. Aktories. 2007. Auto-catalytic cleavage of Clostridium difficile toxins A and B depends on cysteine protease activity. J Biol Chem 282:25314-25321.
17. Elder, G., C. H. Gray, and D. C. Nicholson. 1972. Bile pigment fate in gastrointestinal tract. Semin Hematol 9:71-89.
18. Elliott, B., B. J. Chang, C. L. Golledge, and T. V. Riley. 2007. Clostridium difficile-associated diarrhoea. Intern Med J 37:561-568.
19. Feltis, B. A., S. M. Wiesner, A. S. Kim, S. L. Erlandsen, D. L. Lyerly, T. D. Wilkins, and C. L. Wells. 2000. Clostridium difficile toxins A and B can alter epithelial permeability and promote bacterial paracellular migration through HT-29 enterocytes. Shock 14:629-634.
20. Gasteiger E., H. C., Gattiker A., Duvaud S., Wilkins M. R., Appel R. D., Bairoch A. 2005. Protein identification and analysis tools on the ExPASy server. John M. Walker (ed): The Proteomics Protocols Handbook, Humana Press
21. Genth, H., S. C. Dreger, J. Huelsenbeck, and I. Just. 2008. Clostridium difficile toxins: more than mere inhibitors of Rho proteins. Int J Biochem Cell Biol 40:592-597.
22. Geric, B., M. Rupnik, D. N. Gerding, M. Grabnar, and S. Johnson. 2004. Distribution of Clostridium difficile variant toxinotypes and strains with binary toxin genes among clinical isolates in an American hospital. J Med Microbiol 53:887-894.
23. Gonda, D. K., A. Bachmair, I. Wunning, J. W. Tobias, W. S. Lane, and A. Varshaysky. 1989. Universality and structure of the N-end rule. J Biol Chem 264:16700-16712.
24. Grabowski, G. A., S. Gatt, J. Kruse, and R. J. Desnick. 1984. Human lysosomal beta-glucosidase: kinetic characterization of the catalytic, aglycon, and hydrophobic binding sites. Arch Biochem Biophys 231:144-157.
25. Guruprasad, K., B. V. Reddy, and M. W. Pandit. 1990. Correlation between stability of a protein and its dipeptide composition: a novel approach for predicting in vivo stability of a protein from its primary sequence. Protein Eng 4:155-161.
26. Ho, J. G., A. Greco, M. Rupnik, and K. K. Ng. 2005. Crystal structure of receptor-binding C-terminal repeats from Clostridium difficile toxin A. Proc Natl Acad Sci USA 102:18373-18378.
27. Hofmann, F., C. Busch, U. Prepens, I. Just, and K. Aktories. 1997. Localization of the glucosyltransferase activity of Clostridium difficile toxin B to the N-terminal part of the holotoxin. J Biol Chem 272:11074-11078.
28. Holleran, W. M., Y. Takagi, G. Imokawa, S. Jackson, J. M. Lee, and P. M. Elias. 1992. beta-Glucocerebrosidase activity in murine epidermis: characterization and localization in relation to differentiation. J Lipid Res 33:1201-1209.
29. Just, I., and R. Gerhard. 2004. Large clostridial cytotoxins. Rev Physiol Biochem Pharmacol 152:23-47.
30. Just, I., J. Selzer, M. Wilm, C. von Eichel-Streiber, M. Mann, and K. Aktories. 1995. Glucosylation of Rho proteins by Clostridium difficile toxin B. Nature 375:500-503.
31. Just, I., M. Wilm, J. Selzer, G. Rex, C. von Eichel-Streiber, M. Mann, and K. Aktories. 1995. The enterotoxin from Clostridium difficile (ToxA) monoglucosylates the Rho proteins. J Biol Chem 270:13932-13936.
32. Khan, M. S., B. K. Sridhar, and A. Srinatha. 2010. Development and evaluation of pH-dependent micro beads for colon targeting. Indian J Pharm Sci 72:18-23.
33. Kuehne, S. A., S. T. Cartman, J. T. Heap, M. L. Kelly, A. Cockayne, and N. P. Minton. 2010. The role of toxin A and toxin B in Clostridium difficile infection. Nature.
34. Lanis, J. M., S. Barua, and J. D. Ballard. 2010. Variations in TcdB activity and the hypervirulence of emerging strains of Clostridium difficile. PLoS Pathog 6.
35. Loo, V. G., L. Poirier, M. A. Miller, M. Oughton, M. D. Libman, S. Michaud, A. M. Bourgault, T. Nguyen, C. Frenette, M. Kelly, A. Vibien, P. Brassard, S. Fenn, K. Dewar, T. J. Hudson, R. Horn, P. Rene, Y. Monczak, and A. Dascal. 2005. A predominantly clonal multi-institutional outbreak of Clostridium difficile-associated diarrhea with high morbidity and mortality. N Engl J Med 353:2442-2449.
36. Lyerly, D. M., D. E. Lockwood, S. H. Richardson, and T. D. Wilkins. 1982. Biological activities of toxins A and B of Clostridium difficile. Infect Immun 35:1147-1150.
37. Lyerly, D. M., K. E. Saum, D. K. MacDonald, and T. D. Wilkins. 1985. Effects of Clostridium difficile toxins given intragastrically to animals. Infect Immun 47:349-352.
38. McDonald, L. C., G. E. Killgore, A. Thompson, R. C. Owens, Jr., S. V. Kazakova, S. P. Sambol, S. Johnson, and D. N. Gerding. 2005. An epidemic, toxin gene-variant strain of Clostridium difficile. N Engl J Med 353:2433-2441.
39. Meador, J., 3rd, and R. K. Tweten. 1988. Purification and characterization of toxin B from Clostridium difficile. Infect Immun 56:1708-1714.
40. Monte, M. J., J. J. Marin, A. Antelo, and J. Vazquez-Tato. 2009. Bile acids: chemistry, physiology, and pathophysiology. World J Gastroenterol 15:804-816.
41. Muto, C. A., M. Pokrywka, K. Shutt, A. B. Mendelsohn, K. Nouri, K. Posey, T. Roberts, K. Croyle, S. Krystofiak, S. Patel-Brown, A. W. Pasculle, D. L. Paterson, M. Saul, and L. H. Harrison. 2005. A large outbreak of Clostridium difficile-associated disease with an unexpected proportion of deaths and colectomies at a teaching hospital following increased fluoroquinolone use. Infect Control Hosp Epidemiol 26:273-280.
42. Northfield, T. C., and I. McColl. 1973. Postprandial concentrations of free and conjugated bile acids down the length of the normal human small intestine. Gut 14:513-518.
43. Peters, S. P., P. Coyle, and R. H. Glew. 1976. Differentiation of beta-glucocerebrosidase from beta-glucosidase in human tissues using sodium taurocholate. Arch Biochem Biophys 175:569-582.
44. Pfeifer, G., J. Schirmer, J. Leemhuis, C. Busch, D. K. Meyer, K. Aktories, and H. Barth. 2003. Cellular uptake of Clostridium difficile toxin B. Translocation of the N-terminal catalytic domain into the cytosol of eukaryotic cells. J Biol Chem 278:44535-44541.
45. Poley, J. R., and A. F. Hofmann. 1976. Role of fat maldigestion in pathogenesis of steatorrhea in ileal resection. Fat digestion after two sequential test meals with and without cholestyramine. Gastroenterology 71:38-44.
46. Poutanen, S. M., and A. E. Simor. 2004. Clostridium difficile-associated diarrhea in adults. CMAJ 171:51-58.
47. Radominska-Pandya, A., P. J. Czernik, J. M. Little, E. Battaglia, and P. I. Mackenzie. 1999. Structural and functional studies of UDP-glucuronosyltransferases. Drug Metab Rev 31:817-899.
48. Reineke, J., S. Tenzer, M. Rupnik, A. Koschinski, O. Hasselmayer, A. Schrattenholz, H. Schild, and C. von Eichel-Streiber. 2007. Autocatalytic cleavage of Clostridium difficile toxin B. Nature 446:415-419.
49. Rupnik, M., J. S. Brazier, B. I. Duerden, M. Grabnar, and S. L. Stubbs. 2001. Comparison of toxinotyping and PCR ribotyping of Clostridium difficile strains and description of novel toxinotypes. Microbiology 147:439-447.
50. Rupnik, M., S. Pabst, C. von Eichel-Streiber, H. Urlaub, and H. D. Soling. 2005. Characterization of the cleavage site and function of resulting cleavage fragments after limited proteolysis of Clostridium difficile toxin B (TcdB) by host cells. Microbiology 151:199-208.
51. Russell, J. S. a. D. (ed.). 2000. Molecular Cloning: A Laboratory Manual, 3rd ed. Cold Spring Harbor Laboratory.
52. Sebaihia, M., B. W. Wren, P. Mullany, N. F. Fairweather, N. Minton, R. Stabler, N. R. Thomson, A. P. Roberts, A. M. Cerdeno-Tarraga, H. Wang, M. T. Holden, A. Wright, C. Churcher, M. A. Quail, S. Baker, N. Bason, K. Brooks, T. Chillingworth, A. Cronin, P. Davis, L. Dowd, A. Fraser, T. Feltwell, Z. Hance, S. Holroyd, K. Jagels, S. Moule, K. Mungall, C. Price, E. Rabbinowitsch, S. Sharp, M. Simmonds, K. Stevens, L. Unwin, S. Whithead, B. Dupuy, G. Dougan, B. Barrell, and J. Parkhill. 2006. The multidrug-resistant human pathogen Clostridium difficile has a highly mobile, mosaic genome. Nat Genet 38:779-786.
53. Shikita, M., J. W. Fahey, T. R. Golden, W. D. Holtzclaw, and P. Talalay. 1999. An unusual case of ‘uncompetitive activation’ by ascorbic acid: purification and kinetic properties of a myrosinase from Raphanus sativus seedlings. Biochem J 341 (Pt 3):725-732.
54. Sullivan, N. M., S. Pellett, and T. D. Wilkins. 1982. Purification and characterization of toxins A and B of Clostridium difficile. Infect Immun 35:1032-1040.
55. Tobias, J. W., T. E. Shrader, G. Rocap, and A. Varshaysky. 1991. The N-end rule in bacteria. Science 254:1374-1377.
56. von Eichel-Streiber, C., P. Boquet, M. Sauerborn, and M. Thelestam. 1996. Large clostridial cytotoxins—a family of glycosyltransferases modifying small GTP-binding proteins. Trends Microbiol 4:375-382.
57. von Eichel-Streiber, C., U. Harperath, D. Bosse, and U. Hadding. 1987. Purification of two high molecular weight toxins of Clostridium difficile which are antigenically related. Microb Pathog 2:307-318.
58. Voth, D. E., and J. D. Ballard. 2005. Clostridium difficile toxins: mechanism of action and role in disease. Clin Microbiol Rev 18:247-263.
59. Warny, M., J. Pepin, A. Fang, G. Killgore, A. Thompson, J. Brazier, E. Frost, and L. C. McDonald. 2005. Toxin production by an emerging strain of Clostridium difficile associated with outbreaks of severe disease in North America and Europe. Lancet 366:1079-1084.
60. Babady, N. E., J. Stiles, P. Ruggiero, P. Khosa, D. Huang, S. Shuptar, M. Kamboj, and T. E. Kiehn. 2010. Evaluation of the Cepheid Xpert Clostridium difficile Epi assay for diagnosis of Clostridium difficile infection and typing of the NAP1 strain at a cancer hospital. J Clin Microbiol 48:4519-4524.
61. Dineen, S. S., A. C. Villapakkam, J. T. Nordman, and A. L. Sonenshein. 2007. Repression of Clostridium difficile toxin gene expression by CodY. Mol Microbiol 66:206-219.
62. Dowell, V. R., Jr. 1975. Methods for isolation of anaerobes in the clinical laboratory. Am J Med Technol 41:402-410.
63. Eastwood, K., P. Else, A. Charlett, and M. Wilcox. 2009. Comparison of nine commercially available Clostridium difficile toxin detection assays, a real-time PCR assay for C. difficile tcdB, and a glutamate dehydrogenase detection assay to cytotoxin testing and cytotoxigenic culture methods. J Clin Microbiol 47:3211-3217.
64. Eggertson, L., and B. Sibbald. 2004. Hospitals battling outbreaks of C. difficile. CMAJ 171:19-21.
65. Elliott, B., B. J. Chang, C. L. Golledge, and T. V. Riley. 2007. Clostridium difficile-associated diarrhoea. Intern Med J 37:561-568.
66. George, W. L., V. L. Sutter, D. Citron, and S. M. Finegold. 1979. Selective and differential medium for isolation of Clostridium difficile. J Clin Microbiol 9:214-219.
67. Goorhuis, A., D. Bakker, J. Corver, S. B. Debast, C. Harmanus, D. W. Notermans, A. A. Bergwerff, F. W. Dekker, and E. J. Kuijper. 2008. Emergence of Clostridium difficile infection due to a new hypervirulent strain, polymerase chain reaction ribotype 078. Clin Infect Dis 47:1162-1170.
68. Goorhuis, A., S. B. Debast, L. A. van Leengoed, C. Harmanus, D. W. Notermans, A. A. Bergwerff, and E. J. Kuijper. 2008. Clostridium difficile PCR ribotype 078: an emerging strain in humans and in pigs? J Clin Microbiol 46:1157; author reply 1158.
69. Govind, R., G. Vediyappan, R. D. Rolfe, B. Dupuy, and J. A. Fralick. 2009. Bacteriophage-mediated toxin gene regulation in Clostridium difficile. J Virol 83:12037-12045.
70. Hammond, G. A., and J. L. Johnson. 1995. The toxigenic element of Clostridium difficile strain VPI 10463. Microb Pathog 19:203-213.
71. Hammond, G. A., D. M. Lyerly, and J. L. Johnson. 1997. Transcriptional analysis of the toxigenic element of Clostridium difficile. Microb Pathog 22:143-154.
72. Haslam, S. C., J. M. Ketley, T. J. Mitchell, J. Stephen, D. W. Burdon, and D. C. Candy. 1986. Growth of Clostridium difficile and production of toxins A and B in complex and defined media. J Med Microbiol 21:293-297.
73. Hundsberger, T., V. Braun, M. Weidmann, P. Leukel, M. Sauerborn, and C. von Eichel-Streiber. 1997. Transcription analysis of the genes tcdA-E of the pathogenicity locus of Clostridium difficile. Eur J Biochem 244:735-742.
74. Lyerly, D. M., H. C. Krivan, and T. D. Wilkins. 1988. Clostridium difficile: its disease and toxins. Clin Microbiol Rev 1:1-18.
75. Kyne, L., M. B. Hamel, R Polavaram, and C. P. Kelly. 2002. Health care costs and mortality associated with nosocomial diarrhea due to Clostridium difficile. Clin Infect Dis 34:346-353.
76. Lyerly, D. M., C. J. Phelps, J. Toth, and T. D. Wilkins. 1986. Characterization of toxins A and B of Clostridium difficile with monoclonal antibodies. Infect Immun 54:70-76.
77. Moncrief, J. S., L. A. Barroso, and T. D. Wilkins. 1997. Positive regulation of Clostridium difficile toxins. Infect Immun 65:1105-1108.
78. Noren, T., I. Alriksson, J. Andersson, T. Akerlund, and M. Unemo. 2011. Rapid and sensitive loop-mediated isothermal amplification test for Clostridium difficile detection challenges cytotoxin B cell test and culture as gold standard. J Clin Microbiol 49:710-711.
79. O'Brien, J. A., B. J. Lahue, J. J. Caro, and D. M. Davidson. 2007. The emerging infectious challenge of Clostridium difficile-associated disease in Massachusetts hospitals: clinical and economic consequences. Infect Control Hosp Epidemiol 28:1219-1227.
80. Pothoulakis, C. 2000. Effects of Clostridium difficile toxins on epithelial cell barrier. Ann N Y Acad Sci 915:347-356.
81. Redelings, M. D., F. Sorvillo, and L. Mascola. 2007: Increase in Clostridium difficile-related mortality rates, United States, 1999-2004. Emerg Infect Dis 13:1417-1419.
82. Reller, M. E., C. A. Lema, T. M. Perl, M. Cai, T. L. Ross, K. A. Speck, and K. C. Carroll. 2007. Yield of stool culture with isolate toxin testing versus a two-step algorithm including stool toxin testing for detection of toxigenic Clostridium difficile. J Clin Microbiol 45:3601-3605.
83. Rolfe, R. D., and S. M. Finegold. 1979. Purification and characterization of Clostridium difficile toxin. Infect Immun 25:191-201.
84. Tenover, F. C., S. Novak-Weekley, C. W. Woods, L. R. Peterson, T. Davis, P. Schreckenberger, F. C. Fang, A. Dascal, D. N. Gerding, J. H. Nomura, R. V. Goering, T. Akerlund, A. S. Weissfeld, E. J. Baron, E. Wong, E. M. Marlowe, J. Whitmore, and D. H. Persing. 2010. Impact of strain type on detection of toxigenic Clostridium difficile: comparison of molecular diagnostic and enzyme immunoassay approaches. J Clin Microbiol 48:3719-3724.
85. Thelestam, M., and E. Chaves-Olarte. 2000. Cytotoxic effects of the Clostridium difficile toxins. Curr Top Microbiol Immunol 250:85-96.
86. Ticehurst, J. R., D. Z. Aird, L. M. Dam, A. P. Borek, J. T. Hargrove, and K. C. Carroll. 2006. Effective detection of toxigenic Clostridium difficile by a two-step algorithm including tests for antigen and cytotoxin. J Clin Microbiol 44:1145-1149.
87. Wilkins, T. D., and D. M. Lyerly. 2003. Clostridium difficile testing: after 20 years, still challenging. J Clin Microbiol 41:531-534.
88. Yamakawa, K., S. Kamiya, X. Q. Meng, T. Karasawa, and S. Nakamura. 1994. Toxin production by Clostridium difficile in a defined medium with limited amino acids. J Med Microbiol 41:319-323.

Without further elaboration, it is believed that one skilled in the art can, using the description herein, utilize the present invention to its fullest extent. The embodiments described herein are to be construed as illustrative and not as constraining the remainder of the disclosure in any way whatsoever. While the preferred embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

TABLE 1
Summary of C. difficile toxins A and B purification from crude culture supernatant.

| | PROTEIN AMOUNT | | TOTAL ACTIVITY | | SPECIFIC | | |
|---|---|---|---|---|---|---|---|
| | (μg) | | (Unit)^{a} | | ACTIVITY (U/μg) | | PURIFICATION |
| PURIFICATION STEP | TOXIN A | TOXIN B | TOXIN A | TOXIN B | TOXIN A | TOXIN B | (FOLD)^{b} |
| Crude culture supernatant | 965000 | | 779720 | | 808 | | 1 |
| 150 KDa Concentration | 801000 | | 973215 | | 1215 | | 1.50 |
| DEAE-Sepharose CL-6B | 2230 | 1910 | 1014 | 3085 | 455 | 1615 | 2.56 |
| Sephacryl S-300 | 1410 | 805 | 1158 | 3775 | 821 | 4689 | 6.82 |

^{a}One unit of toxin activity was defined as the amount of the toxins required to cleave one micromole of the PNPG substrate per hour under the experimental conditions.
^{b}Fold purification was calculated using the combined specific activities of toxins A and B.

What is claimed is:

1. A culture medium comprising an agar containing D-cycloserine, cefoxitin, and an indicator-linked substrate for glucosyltransferase.

2. The medium of claim 1 wherein said agar comprises:
a) brain heart infusion;
b) peptic digest of animal tissue;
c) pancreatic digest of gelatin;
d) sodium chloride;
e) dextrose;
f) anhydrous Na₂HPO₄;
g) said D-cycloserine;
h) said cefoxitin;
i) said indicator-linked substrate for glucosyltransferase;
j) 4-methylphenol;
k) defibrinated mammalian blood.

3. The method of claim 1, wherein said medium comprises sodium taurocholate in an amount that does not inhibit cleavage activity of C. difficile toxin.

4. The culture medium of claim 1, wherein the indicator-linked substrate is a chromogenic compound selected from the group consisting of p-nitrophenyl-β-D-glucopyranoside, 4-aminophenyl-α-D-glucopyranoside, 4-aminophenyl-β-D-glucopyranoside, 5-benzyloxy-3-indoxyl-β-D-glucopyranoside, 5-bromo-6-chloro-3-indoxyl-β-D-glucopyranoside, 6-bromo-2-naphthyl-α-D-glucopyranoside, 6-chloro-3-indoxyl-α-D-glucopyranoside, 6-chloro-3-indoxyl-N-acetyl-beta-D-glucosaminide, 5-bromo-3-indolyl-β-D-galactopyranoside, 5-bromo-3-indoxyl-β-D-galactopyranoside, and 5-bromo-4-chloro-3-indoxyl-β-D-galactopyranoside.

5. The culture medium of claim 4, wherein said chromogenic compound is 5-bromo-3-indolyl-β-D-galactopyranoside.

6. The culture medium of claim 1, wherein said indicator is a fluorescent or chemiluminescent molecule.

7. An assay medium for measuring glucosyltransferase activity of C. difficile toxin comprising:
a) a sample suspected of containing a toxin-producing C. difficile;
b) an aqueous buffer that maintains the medium at a pH in the range of about 7 to about 9;
c) monovalent and/or divalent salt; and
d) an indicator-linked substrate for glucosyltransferase.

8. The assay medium of claim 7, wherein the indicator-linked substrate is a chromogenic compound selected from the group consisting of p-nitrophenyl-β-D-glucopyranoside, p-nitrophenyl-α-D-glucopyranoside, 4-aminophenyl-α-D-glucopyranoside, 4-aminophenyl-β-D-glucopyranoside, 5-benzyloxy-3-indoxyl-β-D-glucopyranoside, 5-bromo-6-chloro-3-indoxyl-β-D-glucopyranoside, 6-bromo-2-naphthyl-α-D-glucopyranoside, 6-chloro-3-indoxyl-α-D-glucopyranoside, 6-chloro-3-indoxyl-N-acetyl-beta-D-glucosaminide, 5-bromo-3-indoxyl-β-D-galactopyranoside, and 5-bromo-4-chloro-3-indoxyl-β-D-galactopyranoside.

9. The assay medium of claim 8, wherein said chromogenic compound is p-nitrophenyl-β-D-glucopyranoside (PNPG)

10. A method of detecting the presence of a toxin-producing C. difficile, comprising:
a) contacting a sample suspected of containing a toxin-producing strain of C. difficile with a medium comprising an indicator-linked substrate for glucosyltransferase;
b) incubating the resulting culture in an anaerobic environment for a time sufficient to allow cleavage of the indicator-substrate link if a C. difficile toxin having glucosyltransferase activity is present; and
c) detecting cleavage of the indicator-substrate link in the incubated culture from b); and
d) determining that a toxin-producing strain of C. difficile is present in the sample based upon detected cleavage in c).

11. The method of claim 10, wherein, in c), said detecting comprises monitoring the color of bacterial colonies in the culture prior to and during said incubation in b).

12. The method of claim 11, wherein toxin-producing C. difficile colonies appear blue while non-toxin producing colonies remain pale white.

13. The method of claim 10, wherein the medium is as defined in any of claims 1-6.

14. The method of claim 10, wherein in c), detecting cleavage of the indicator-substrate link comprises quantifying the level of said cleavage.

15. The method of claim 10, wherein, in c), said detecting comprises measuring a change in color of a supernatant removed from the culture after said incubation in b).

16. The method of claim 10, wherein, in c), said detecting comprises measuring a change in absorbance of electromagnetic radiation at a predetermined wavelength by a supernatant removed from the culture after said incubation.

17. The method of claim 9, wherein, in c), said detecting comprises e) determining a level of toxin-producing C. difficile in said sample based on measurement of glucosyltransferase activity in said sample.

18. The method of claim 10, wherein said indicator-linked substrate is a chromogenic substrate selected from the group consisting of p-nitrophenyl-α-D-glucopyranoside, p-nitrophenyl-β-D-glucopyranoside; 4-aminophenyl-α-D-glucopyranoside, 4-aminophenyl-β-D-glucopyranoside, 5-benzyloxy-3-indoxyl-β-D-glucopyranoside, 5-bromo-6-chloro-3-indoxyl-β-D-glucopyranoside, 6-bromo-2-naphthyl-α-D-glucopyranoside, 6-chloro-3-indoxyl-α-D-glucopyranoside, 6-chloro-3-indoxyl-N-acetyl-beta-D-glucosaminide, 5-bromo-3-indoxyl-β-D-galactopyranoside, and 5-bromo-4-chloro-3-indoxyl-β-D-galactopyranoside.

19. The method of claim 18, wherein said chromogenic substrate is p-nitrophenyl-β-D-glucopyranoside (PNPG).

20. A method of identifying the presence of an active C. difficile toxin, comprising:
a) incubating a sample suspected of containing a C. difficile toxin in an assay medium comprising an aqueous buffer that maintains the medium at a pH in the range of about 7 to about 9, a monovalent and/or divalent salt, and an indicator-linked substrate for glucosyltransferase; and
b) measuring glucosyltransferase activity of the sample based on detection of cleavage of the indicator-substrate link in a).

21. The method of claim 20, wherein a) comprises combining 100 μl of the sample with 200 μl of a reagent comprising about 2 to about 10 mM PNPG, 50 mM Tris-HCl (pH 7.4), 50 mM NaCl, and 100 μM MnCl₂;
in b), said incubating comprises incubating the resulting culture at 37° C. for 1-4 hrs and adding 40 μl of 3 M Na₂CO₃, and
in c), said determining comprises measuring a change in electromagnetic radiation absorbance between 400-500 nm in a supernatant removed from the culture in b).

22. The method of claim 1, wherein said medium contains an oxidizing agent such as dimethyl sulfoxide.

23. A method of screening a population of individuals for infection by toxin-producing C. Difficile, comprising
a) measuring in a biological sample from each individual glucosyltransferase activity based on detection of enzymatic cleavage of indicator from an indicator-linked substrate for glucosyltransferase; and
(b) administering a therapeutic treatment for toxin-producing C. difficile to patients with samples that tested positive for glucosyltransferase activity.

24. The method of claim 23 wherein said biological sample is a supernatant obtained from a cell culture of a biological specimen obtained from the individual, wherein the cell culture is prepared by
(i) contacting the specimen suspected of containing a toxin-producing strain of C. difficile with a medium comprising an indicator-linked substrate for glucosyltransferase;
ii) incubating the resulting culture in an anaerobic environment for a time sufficient to allow cleavage of the indicator-substrate link if a C. difficile toxin having glucosyltransferase activity is present.

25. A screening method to identify a substance that inhibits the pathogenesis of C. difficile toxin, comprising:
a) combining a test substance with (i) a sample containing toxin-producing C. difficile, (ii) an anaerobic incubation medium comprising a buffer that maintains the medium at a pH in the range of about 7 to about 9, monovalent and/or divalent salt, and (iii) an indicator-linked substrate for glucosyltransferase;
b) incubating the resulting combination in an anaerobic environment for a sufficient time to allow cleavage of the indicator-linked substrate in the presence of toxin, but absence of the test substance;
c) determining whether the test substance inhibits cleavage of the indicator from the substrate based upon a detected level of free indicator in b) relative to a control level of free indicator obtained in the absence of the test substance.

26. The screening method of claim 25, wherein, in c), said determining comprises detecting a lack of color change in the incubation medium in b).

27. The screening method of claim 25, wherein, in c), said determining comprises detecting a fluorescence change in the incubation medium in b).

28. The screening method of claim 25, wherein in c), said determining comprises detecting a lack of change in absorbance at a predetermined wavelength by the incubation medium in b).
